(19) United States
(12) Patent Application Publication
(10) Pub. No.: US 2022/0095666 A1
(43) Pub. Date: Mar. 31, 2022

(54) NUTRITIONAL COMPOSITIONS CONTAINING BUTYRATE AND USES THEREOF

(71) Applicant: MEAD JOHNSON NUTRITION COMPANY, Evansville, IN (US)

(72) Inventors: Marieke H. Schoemaker, Rhenen (NL); Teartse Tim Lambers, Nijmegen (NL); Gabriele Gross, Nijmegen (NL); Eric A.F. van Tol, Arnhem (NL); Shay Phillips, Oakland City, IN (US)

(73) Assignee: MEAD JOHNSON NUTRITION COMPANY, Evansville, IN (US)

(21) Appl. No.: 17/500,107

(22) Filed: Oct. 13, 2021

Related U.S. Application Data
(62) Division of application No. 15/376,330, filed on Dec. 12, 2016, now abandoned.

(51) Int. Cl. A23L 33/00; A23L 33/135; A23L 33/12; A23L 33/10; A23L 33/17; A23L 33/15; A23L 33/155; A23L 33/16; A61K 31/19; A61K 31/716; A61K 31/59; A61K 38/01; A61P 3/08; A61P 3/02; A61K 35/747; A61K 45/06; A61K 33/26; A61P 3/00; A61P 3/04; A61K 31/047; A61K 31/202; A23L 33/18; A61K 33/30

(52) U.S. Cl. CPC A23L 33/40; A23Y 2220/73; A23L 33/12; A23L 33/10; A23L 33/17; A23L 33/15; A23L 33/155; A23L 33/16; A61K 31/19; A61K 31/716; A61K 31/59; A61K 38/01; A61P 3/08; A61P 3/02; A61K 35/747; A61K 45/06; A61K 33/26; A61P 3/00; A61P 3/04; A61K 31/047; A61K 31/202; A23L 33/18; A61K 33/30; A23V 2002/00; A23L 33/135

(57) ABSTRACT

Provided are nutritional compositions containing dietary butyrate. Further disclosed are methods for reducing the incidence of obesity and metabolic syndrome in a target subject via administering the nutritional composition containing dietary butyrate to the target subject. Further provided are methods for improving adipose tissue functioning in a target subject.

### TECHNICAL FIELD

The present disclosure relates generally to nutritional compositions comprising dietary butyrate and uses thereof. The nutritional compositions are suitable for administration to pediatric subjects. Further, disclosed are methods for reducing the incidence of obesity and metabolic syndrome in target subjects. Additionally, disclosed are methods for maintaining healthy weight and metabolism in target subjects. Further disclosed are methods for improving adipose tissue function and improving the quality of adipose tissue in target subjects. The disclosed nutritional compositions may provide additive and/or synergistic beneficial health effects.

### BACKGROUND ART

Obesity represents a major global health problem that is frequently associated with chronic diseases such as type-2 diabetes. Furthermore, obesity is the hallmark of metabolic syndrome. Childhood overweight and obesity has increased substantially in the past two decades causing certain cardiovascular, muscle/skeletal, and endocrine complications to start in early childhood. Furthermore, there is an increase in the incidence rates of non-alcoholic fatty liver disease and cognitive impairment in children. Accordingly, there exists the need to provide an infant formula or nutritional composition that is capable of reducing the incidence of obesity and reducing the incidence of symptoms and chronic diseases associated with metabolic syndrome in infants and children.

Thus, provided herein are nutritional compositions containing dietary butyrate in combination with other nutrients that promote a healthy body weight and reduce the risk of obesity when administered to a target subject, such as an infant. Further provided are compositions for improving adipose tissue function and improving the quality of adipose tissue in target subjects in formula-fed infants.

### BRIEF SUMMARY

Briefly, the present disclosure is directed, in an embodiment, to a nutritional composition that includes dietary butyrate. In some embodiments, the dietary butyrate may be provided in the form of sodium butyrate, butyrate triglycerides, encapsulated butyrate, or enriched lipid fractions from milk. In some embodiments, the nutritional composition includes dietary butyrate in combination with long chain polyunsaturated fatty acids, such as docosahexaenoic acid and/or arachidonic acid, one or more probiotics, such as Lactobacillus rhamnosus GG, phosphatidylethanolamine (PE), sphingomyelin, inositol, vitamin D, and combinations thereof.

The present disclosure further provides methods for promoting and or maintaining a healthy weight in pediatric subjects by administering the nutritional composition disclosed herein to the pediatric subject.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### BRIEF DESCRIPTION OF THE DRAWINGS

FIG. 1 illustrates an example chromatogram of ethyl acetate solvent blank.

FIG. 2 illustrates an example chromatogram of an internal standard at 15 and butyric acid at 50 μg/mL.

FIG. 3 illustrates an example chromatogram of NP H₂O blank through the sample preparation.

FIG. 4A illustrates a chromatogram of a human milk sample

FIG. 4B illustrates the same chromatogram of a human milk sample showing the internal standard and butyric acid (BA) peaks.

FIG. 5 is a sample sequence setup template.

FIG. 6 illustrates a schematic process flow chart for the procedure outlined in Example 4.

### DETAILED DESCRIPTION

Reference now will be made in detail to the embodiments of the present disclosure, one or more examples of which are set forth herein below. Each example is provided by way of explanation of the nutritional composition of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are apparent from the following detailed description. It is to be understood by one of ordinary skill in the art that the present discussion is a description of exemplary embodiments only and is not intended as limiting the broader aspects of the present disclosure.

The present disclosure relates generally to nutritional compositions comprising dietary butyrate in combination with other nutrients disclosed herein. Additionally, the disclosure relates to methods for maintaining healthy weight, reducing the incidence of obesity, reducing the incidence of metabolic syndrome, improving adipose tissue function, and improving the quality of adipose tissue in target subjects.

“Nutritional composition” means a substance or formulation that satisfies at least a portion of a subject's nutrient requirements. The terms “nutritional (s)”, “nutritional formula (s)”, “enteral nutritional (s)”, and “nutritional supplement(s)” are used as non-limiting examples of nutritional composition(s) throughout the present disclosure. Moreover, “nutritional composition(s)” may refer to liquids, powders, gels, pastes, solids, tablets, capsules, concentrates, suspensions, or ready-to-use forms of enteral formulas, oral formulas, formulas for infants, formulas for pediatric subjects, formulas for children, growing-up milks and/or formulas for adults.

“Pediatric subject” means a human less than 13 years of age. In some embodiments, a pediatric subject refers to a human subject that is between birth and 8 years old. In other embodiments, a pediatric subject refers to a human subject between 1 and 6 years of age. In still further embodiments, a pediatric subject refers to a human subject between 6 and 12 years of age. The term “pediatric subject” may refer to infants (preterm or fullterm) and/or children, as described below.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes low birth weight infants, very low birth weight infants, and preterm infants. “Preterm” means an infant born before the end of the 37th week of gestation. “Full term” means an infant born after the end of the 37th week of gestation.

“Child” means a subject ranging in age from 12 months to about 13 years. In some embodiments, a child is a subject between the ages of 1 and 12 years old. In other embodiments, the terms “children” or “child” refer to subjects that are between one and about six years old, or between about seven and about 12 years old. In other embodiments, the terms “children” or “child” refer to any range of ages between 12 months and about 13 years.

“Infant formula” means a composition that satisfies at least a portion of the nutrient requirements of an infant. In the United States, the content of an infant formula is dictated by the federal regulations set forth at 21 C.F.R. Sections 100, 106, and 107.

The term “medical food” refers enteral compositions that are formulated or intended for the dietary management of a disease or disorder. A medical food may be a food for oral ingestion or tube feeding (nasogastric tube), may be labeled for the dietary management of a specific medical disorder, disease or condition for which there are distinctive nutritional requirements, and may be intended to be used under medical supervision.

The term “peptide” as used herein describes linear molecular chains of amino acids, including single chain molecules or their fragments. The peptides described herein include no more than 50 total amino acids. Peptides may further form oligomers or multimers consisting of at least two identical or different molecules. Furthermore, peptidomimetics of such peptides where amino acid (s) and/or peptide bond (s) have been replaced by functional analogs are also encompassed by the term “peptide”. Such functional analogues may include, but are not limited to, all known amino acids other than the 20 gene-encoded amino acids such as selenocysteine.

The term “peptide” may also refer to naturally modified peptides where the modification is effected, for example, by glycosylation, acetylation, phosphorylation and similar modification which are well known in the art. In some embodiments, the peptide component is distinguished from a protein source also disclosed herein. Further, peptides may, for example, be produced recombinantly, semi-synthetically, synthetically, or obtained from natural sources such as after hydrolysation of proteins, including but not limited to casein, all according to methods known in the art.

The term “molar mass distribution” when used in reference to a hydrolyzed protein or protein hydrolysate pertains to the molar mass of each peptide present in the protein hydrolysate. For example, a protein hydrolysate having a molar mass distribution of greater than 500 Daltons means that each peptide included in the protein hydrolysate has a molar mass of at least 500 Daltons. Accordingly, in some embodiments, the peptides disclosed in Table 3 and Table 4 are derived from a protein hydrolysate having a molar mass distribution of greater than 500 Daltons. To produce a protein hydrolysate having a molar mass distribution of greater than 500 Daltons, a protein hydrolysate may be subjected to certain filtering procedures or any other procedure known in the art for removing peptides, amino acids, and/or other proteinaceous material having a molar mass of less than 500 Daltons. For the purposes of this disclosure, any method known in the art may be used to produce the protein hydrolysate having a molar mass distribution of greater than 500 Dalton.

The term “protein equivalent” or “protein equivalent source” includes any protein source, such as soy, egg, whey, or casein, as well as non-protein sources, such as peptides or amino acids. Further, the protein equivalent source can be any used in the art, e.g., nonfat milk, whey protein, casein, soy protein, hydrolyzed protein, peptides, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate), soy bean proteins, and any combinations thereof. The protein equivalent source can, in some embodiments comprise hydrolyzed protein, including partially hydrolyzed protein and extensively hydrolyzed protein. The protein equivalent source may, in some embodiments, include intact protein. More particularly, the protein source may include a) about 20% to about 80% of the peptide component described herein, and b) about 20% to about 80% of an intact protein, a hydrolyzed protein, or a combination thereof.

The term “protein equivalent source” also encompasses free amino acids. In some embodiments, the amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In some embodiments, the amino acids may be branched chain amino acids. In certain other embodiments, small amino acid peptides may be included as the protein component of the nutritional composition. Such small amino acid peptides may be naturally occurring or synthesized.

“Fractionation procedure” includes any process in which a certain quantity of a mixture is divided up into a number of smaller quantities known as fractions. The fractions may be different in composition from both the mixture and other fractions. Examples of fractionation procedures include but are not limited to, melt fractionation, solvent fractionation, supercritical fluid fractionation and/or combinations thereof.

“Milk fat globule membrane” includes components found in the milk fat globule membrane including but not limited to milk fat globule membrane proteins such as Mucin 1, Butyrophilin, Adipophilin, CD36, CD14, Lactadherin (PAS6/7), Xanthine oxidase and Fatty Acid binding proteins etc. Additionally, “milk fat globule membrane” may include phospholipids, cerebrosides, gangliosides, sphingomyelins, and/or cholesterol.

The term “growing-up milk” refers to a broad category of nutritional compositions intended to be used as a part of a diverse diet in order to support the normal growth and development of a child between the ages of about 1 and about 6 years of age.

“Milk” means a component that has been drawn or extracted from the mammary gland of a mammal. In some embodiments, the nutritional composition comprises components of milk that are derived from domesticated ungulates, ruminants or other mammals or any combination thereof.

“Nutritionally complete” means a composition that may be used as the sole source of nutrition, which would supply essentially all of the required daily amounts of vitamins, minerals, and/or trace elements in combination with proteins, carbohydrates, and lipids. Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

A nutritional composition that is “nutritionally complete” for a full term infant will, by definition, provide qualitatively and quantitatively adequate amounts of all carbohydrates, lipids, essential fatty acids, proteins, essential amino acids, conditionally essential amino acids, vitamins, minerals, and energy required for growth of the full term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

“Exogenous butyrate” or “dietary butyrate” each refer to butyrate or butyrate derivatives which are intentionally included in the nutritional composition of the present disclosure itself, rather than generated in the gut.

“Endogenous butyrate” or “butyrate from endogenous sources” each refer to butyrate present in the gut as a result of ingestion of the disclosed composition that is not added as such, but is present as a result of other components or ingredients of the composition; the presence of such other components or ingredients of the composition stimulates butyrate production in the gut.

“Probiotic” means a microorganism with low or no pathogenicity that exerts a beneficial effect on the health of the host.

The term “non-viable probiotic” means a probiotic wherein the metabolic activity or reproductive ability of the referenced probiotic has been reduced or destroyed. More specifically, “non-viable” or “non-viable probiotic” means non-living probiotic microorganisms, their cellular components and/or metabolites thereof. Such non-viable probiotics may have been heat-killed or otherwise inactivated. The “non-viable probiotic” does, however, still retain, at the cellular level, its cell structure or other structure associated with the cell, for example exopolysaccharide and at least a portion its biological glycol-protein and DNA/RNA structure and thus retains the ability to favorably influence the health of the host. Contrariwise, the term “viable” refers to live microorganisms. As used herein, the term “non-viable” is synonymous with “inactivated”.

“Prebiotic” means a non-digestible food ingredient that beneficially affects the host by selectively stimulating the growth and/or activity of one or a limited number of bacteria in the digestive tract that can improve the health of the host.

“Phospholipids” means an organic molecule that contains a diglyceride, a phosphate group and a simple organic molecule. Examples of phospholipids include but are not limited to, phosphatidic acid, phosphatidylethanolamine, phosphatidylcholine, phosphatidylserine, phsphatidylinositol, phosphatidylinositol phosphate, phosphatidylinositol biphosphate and phosphatidylinositol triphosphate, ceramide phosphorylcholine, ceramide phosphorylethanolamine and ceramide phosphoryl glycerol. This definition further includes sphingolipids such as sphingomyelin. Glycosphingolipds are quantitatively minor constituents of the MFGM, and consist of cerebrosides (neutral glycosphingolipids containing uncharged sugars) and gangliosides. Gangliosides are acidic glycosphingolipids that contain sialic acid (N-acetylneuraminic acid (NANA)) as part of their carbohydrate moiety. There are various types of gangliosides originating from different synthetic pathways, including GM3, GM2, GM1a, GD1a, GD3, GD2, GD1b, Glib and GQ1b (Fujiwara et al., 2012). The principal gangliosides in milk are GM3 and GD3 (Pan & Izumi, 1999). The different types of gangliosides vary in the nature and length of their carbohydrate side chains, and the number of sialic acid attached to the molecule.

“Alpha-lipoic acid”, abbreviated “ALA” herein, refers to an organosulfur compound derived from octanoic acid having the molecular formula C₈H₁₄S₂O₂. Generally, ALA contains two sulfur atoms attached via a disulfide bond. Alpha-lipoic acid is synonymous with lipoic acid, abbreviated “LA”, and the two terms and abbreviations may be used interchangeable herein.

As used herein “sulforaphane” includes any known isomers of sulforaphane including but not limited to L-sulforaphane. In some embodiments, sulforaphane may include only L-sulforaphane while, in other embodiments, the reference to sulforaphane may include L-sulforaphane, D-sulforaphane, any other suitable isomer of sulforaphane, and any combinations thereof. Accordingly, the term sulforaphane as used herein includes any isomers of sulforaphane including, but not limited to, stereoisomers, optical isomers, structural isomers, enantiomers, geometric isomers, and combinations thereof.

The nutritional composition of the present disclosure may be substantially free of any optional or selected ingredients described herein, provided that the remaining nutritional composition still contains all of the required ingredients or features described herein. In this context, and unless otherwise specified, the term “substantially free” means that the selected composition may contain less than a functional amount of the optional ingredient, typically less than 0.1% by weight, and also, including zero percent by weight of such optional or selected ingredient.

All percentages, parts and ratios as used herein are by weight of the total composition, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

The present disclosure is directed to nutritional compositions including dietary butyrate. Non-limiting examples of butyrate for use herein include butyric acid, butyrate salts, glycerol esters of butyric acid, and amide derivatives of amino acids. The nutritional compositions may further include a carbohydrate source, a protein source, and a fat or lipid source. In some embodiments, the nutritional compositions may include a component capable of stimulating endogenous butyrate production; in other embodiments, the nutritional compositions may include both dietary and endogenous butyrate.

The benefit to providing dietary butyrate in combination with selected nutrients herein is healthy weight development and metabolism, in particular improving adipose tissue function and quality. Furthermore, providing dietary butyrate in combination with selected nutrients may provide anti-inflammatory properties, such as a reduction in the inflammatory processes in fat tissues, the liver, and the brain.

Indeed, dietary butyrate may affect energy homeostasis, glucose metabolism, and insulin sensitivity. Dietary supplementation with dietary butyrate may prevent the developments of diet-induced insulin resistance and improve insulin sensitivity, thus promoting healthy metabolic programming and reducing the risk of metabolic syndrome. Further, providing dietary butyrate may reduce insulin resistance and reduce obesity-associated inflammation. Without being bound by any particular theory, mechanistically dietary butyrate acts through promotion of mitochondrial energy expenditure and modulation of the inflammatory response. These mechanisms may be involved in maintaining healthy weight during infancy and pediatric development.

In certain embodiments, the dietary butyrate is incorporated into a nutritional composition that is an infant formula. Currently, many infant formulas are not formulated with dietary butyrate or are not formulated with effective amounts of dietary butyrate for providing a beneficial health effect once administered to the infant. One reason that infant formulas include little to no dietary butyrate is due to the unpleasant organoleptic properties exhibited by the nutritional composition when butyrate compounds are incorporated into the nutritional composition. For example, many butyrate compounds exhibit an odor that makes consuming the nutritional composition in which they are incorporated an unpleasant experience. Furthermore, the pediatric and infant population will not readily consume nutritional products having an unpleasant odor, taste, and/or mouthfeel. Accordingly, there exists a need for a nutritional composition formulated for administration to a pediatric subject or an infant that provides butyrate yet does not have diminished organoleptic properties. The incorporation of the dietary butyrate compounds disclosed herein into pediatric and infant nutritional compositions will provide butyrate while still providing a pleasant sensory experience.

Accordingly, given that dietary butyrate is not supplemented in effective levels in infant formula, many formula-fed infants may not obtain enough butyrate through diet in comparison to breast-fed infants. Accordingly, providing the dietary butyrate in an infant formula and administering the formula to a pediatric subject ensures that certain risk factors for cardiovascular disease and metabolic syndrome may be further reduced in formula-fed infants as compared to breast-fed infants. Furthermore, providing the compositions herein to an infant or child may strengthen the capacity and flexibility of fat tissues.

In some embodiments, the nutritional composition includes a source of dietary butyrate that is present in an amount of from about 0.01 mg/100 Kcal to about 300 mg/100 Kcal. In some embodiments, the nutritional composition includes a source of dietary butyrate that is present in an amount of from about 0.1 mg/100 Kcal to about 300 mg/100 Kcal. In some embodiments, the nutritional composition includes a source of dietary butyrate that is present in an amount of from about 0.1 mg/100 Kcal to about 300 mg/100 Kcal. In some embodiments, the nutritional composition includes a source of dietary butyrate that is present in an amount of from about 1 mg/100 Kcal to about 275 mg/100 Kcal. In some embodiments, the nutritional composition includes a source of dietary butyrate that is present in an amount of from about 5 mg/100 Kcal to about 200 mg/100 Kcal. In some embodiments, the nutritional composition includes a source of dietary butyrate that is present in an amount of from about 10 mg/100 Kcal to about 150 mg/100 Kcal. In some embodiments the amount of butyrate is from about 0.6 mg/100 kcal to about 6.1 mg per 100 kcal.

In some embodiments, the nutritional composition includes a source of dietary butyrate that is present in an amount based on the weight percentage of total fat. Accordingly, in some embodiments the nutritional composition includes from about 0.2 mg to about 57 mg of dietary butyrate per gram of fat in the nutritional composition. In some embodiments, the nutritional compositions includes from about 1 mg to about 50 mg of dietary butyrate per gram of fat in the nutritional composition. Still, in some embodiments the nutritional composition includes from about 5 mg to about 40 mg of dietary butyrate per gram of fat in the nutritional composition. In certain embodiments, the nutritional composition includes from about 10 mg to about 30 mg of dietary butyrate per gram of fat in the nutritional composition.

In some embodiments, the nutritional composition includes a source of dietary butyrate that is present in an amount based on a liter of formula. In some embodiments, the nutritional composition includes from about 0.6 mg to about 2100 mg of dietary butyrate per Liter of nutritional composition. In some embodiments, the nutritional composition includes from about 2 mg to about 2000 mg of dietary butyrate per Liter of nutritional composition. In some embodiments, the nutritional composition includes from about 10 mg to about 1800 mg of dietary butyrate per Liter of nutritional composition. In some embodiments, the nutritional composition includes from about 25 mg to about 1600 mg of dietary butyrate per Liter of nutritional composition. In some embodiments, the nutritional composition includes from about 40 mg to about 1400 mg of dietary butyrate per Liter of nutritional composition. In some embodiments, the nutritional composition includes from about 50 mg to about 1200 mg of dietary butyrate per Liter of nutritional composition. In some embodiments, the nutritional composition includes from about 100 mg to about 1000 mg of dietary butyrate per Liter of nutritional composition.

In some embodiments the dietary butyrate is provided by one or more of the following: butyric acid; butyrate salts, including sodium butyrate, potassium butyrate, calcium butyrate, and/or magnesium butyrate; glycerol esters of butyric acid; and/or amide derivative of butyric acid.

The dietary butyrate can be supplied by any suitable source known in the art. Non-limiting sources of dietary butyrate includes animal source fats and derived products, such as but not limited to milk, milk fat, butter, buttermilk, butter serum, cream; microbial fermentation derived products, such as but not limited to yogurt and fermented buttermilk; and plant source derived seed oil products, such as pineapple and/or pineapple oil, apricot and/or apricot oil, barley, oats, brown rice, bran, green beans, legumes, leafy greens, apples, kiwi, oranges. In some embodiments, the dietary butyrate is synthetically produced. In embodiments where the dietary butyrate is synthetically produced, the chemical structure of the dietary butyrate may be modified as necessary. Further, the dietary butyrate produced synthetically can be purified by any means known in the art to produce a purified dietary butyrate additive that can be incorporated into the nutritional compositions disclosed herein. The dietary butyrate may be provided by dairy lipids and/or triglyceride bound forms of butyrate.

In some embodiments, the dietary butyrate may be provided in an encapsulated form. In certain embodiments, the encapsulation of the dietary butyrate may provide for longer shelf-stability and may provide for improved organoleptic properties of the nutritional composition. For example, in some embodiments, the dietary butyrate may be encapsulated or coated by the use of, or combination of, fat derived materials, such as mono- and di-glycerides; sugar and acid esters of glycerides; phospholipids; plant, animal and microbial derived proteins and hydrocolloids, such as starches, maltodextrins, gelatin, pectins, glucans, caseins, soy proteins, and/or whey proteins.

The dietary butyric acid may also be provided in a coated form. For example, coating certain glycerol esters of butyric acids with fat derived materials, such as mono- and di-glycerides; sugar and acid esters of glycerides; phospholipids; plant, animal and microbial derived proteins and hydrocolloids, such as starches, maltodextrins, gelatin, pectins, glucans, caseins, soy proteins, and/or whey proteins may improve the shelf-stability of the dietary butyrate and may further improve the overall organoleptic properties of the nutritional composition.

In certain embodiments, the dietary butyrate comprises alkyl, and or glycerol esters of butyric acid. Glycerol esters of butyric acid may offer minimal complexity when formulated and processed in the nutritional composition. Additionally, glycerol esters of butyric acid may improve the shelf life of the nutritional composition including dietary butyrate an may further have a low impact on the sensory attributes of the finished product.

The dietary butyrate comprises amide derivatives of butyric acid in some embodiments. Generally, these amide derivatives of butyric acid are a solid, odorless, and tasteless form and are more stable than certain butyric acid esters at gastric pH. Further, the amide derivatives of butyric acid are able to release the corresponding acid by alkaline hydrolysis in the small and large intestine, thereby allowing for absorption of the dietary butyrate.

In some embodiments, the dietary butyrate may comprise butyrate salts, for example, sodium butyrate, potassium butyrate, calcium butyrate, magnesium butyrate, and combinations thereof. In some embodiments, the use of selected dietary butyrate salts may improve intestinal health when provided to target subjects. In certain embodiments, dietary butyrate comprises a suitable butyrate salt that has been coated with one or more fats or lipids. In certain embodiments wherein the dietary butyrate comprises a fat coated butyrate salt, the nutritional composition may be a dry-powdered composition into which the dietary butyrate is incorporated.

In some embodiments, the dietary butyrate may comprise any of the butyrate compounds disclosed herein that are formulated to be in complex form with chitosan or one or cyclodextrins. For example, cyclodextrins are cyclic oligosaccharides composed of six (a-cyclodextrin), seven (β-cyclodextrin), or eight (gamma-cyclodextrin) units of a-1,4-glucopyranose. Cyclodextrins are further characterized by a hydrophilic exterior surface and a hydrophobic core. Without being bound by any particular theory, the aliphatic butyrate chain would form a complex with the cyclodextrin core, thus increasing its molecular weight and, thus, reducing the volatility of the butyrate compound. Accordingly, the bioavailability of dietary butyrate may be improved when the dietary butyrate includes butyrate compounds in complex form with one or more cyclodextrins. Further, cyclodextrins are bulky hydrophobic molecules that are resistant to stomach acid as well as gastrointestinal enzymes, thus administration of the butyrate-cyclodextrin complex as described herein would promote absorption of the dietary butyrate in the small intestines.

In some embodiments the dietary butyrate is provided from an enriched lipid fraction derived from milk. For example, bovine milk fat has a butyric acid content that may be 20 times higher than the butyric acid content in human milk fat. Furthermore, among the short chain fatty acids (“SCFAs”) present in human milk, i.e. fatty acids having a carbon chain length from 4 to 12, butyric acid (C4) is one of the most predominant in bovine milk. As such, bovine milk fat and/or enriched fractions of bovine milk fat may be included in a nutritional composition to provide dietary butyrate.

In embodiments where the dietary butyrate is provided by an enriched lipid fraction derived from milk the enriched lipid fraction derived from milk may be produced by any number of fractionation techniques. These techniques include but are not limited to melting point fractionation, organic solvent fractionation, super critical fluid fractionation, and any variants and combinations thereof.

Furthermore, mixtures that may be subjected to the fractionation procedures to produce the enriched lipid fraction include, but are not limited to, bovine whole milk, bovine cream, caprine milk, ovine milk, yak milk, and/or mixtures thereof. In a preferred embodiment the milk mixture used to create the enriched lipid fraction is bovine milk.

In addition to providing dietary butyrate, the enriched lipid fraction may comprise an one of the following ingredients: saturated fatty acids; trans-fatty acids; branched-chain fatty acids (“BCFAs”), including odd-branched chain fatty acids (“OBCFAs”); conjugated linoleic acid (“CLA”); monounsaturated fatty acids; polyunsaturated fatty acids; cholesterol; phospholipids; and milk fat globule membrane, including milk fat globule membrane protein.

In some embodiments the enriched lipid fraction includes, per 100 Kcal, one or more of the following:

from about 0.1 g to 8.0 g of saturated fatty acids;

from about 0.2 g to 7.0 g trans-fatty acids;

from about 0.003 g to about 6.1 g branched-chain fatty acids;

from about 0.026 g to about 2.5 g conjugated linoleic acid;

from about 0.8 g to about 2.5 g monounsaturated fatty acids;

from about 2.3 g to about 4.4 g polyunsaturated fatty acids;

from about 100 mg to about 400 mg of cholesterol;

from about 50 mg to about 400 mg of phospholipids; and/or from about 10 mg to about 500 mg of milk fat globule membrane.

The following example illustrates a milk fat fraction having an enriched concentration of butyric acid (C4) that may be produced by a fractionation procedure.

### Example 1

Illustrated in Table 1 below is a lipid profile of fractionated milk fat produced by super critical carbon extraction fractionation procedure and by melt-fractionation.

TABLE 1
Milk Fat composition (g fatty acid /100 g TOTAL fatty acids)

| | | |
|---|---|---|
| AMF | SCCO 2 | MeltFrac 10 C. |

| | | | |
|---|---|---|---|
| C 4:0 | 3.9 | 6.0 | 4.7 |
| C 6:0 | 2.5 | 3.3 | 2.9 |
| C 8:0 | 1.4 | 1.9 | 1.8 |
| C 10:0 | 3.1 | 3.9 | 3.8 |
| C 12:0 | 4.2 | 4.1 | 4.8 |
| C 14:0 | 11.4 | 12.2 | 10.9 |
| C 14:1 | 1.1 | 1.0 | 1.3 |
| C 15:0 | 1.1 | 1.0 | 0.9 |
| C 16:0 | 29.4 | 29.6 | 22.3 |
| C 16:1 | 1.9 | 1.4 | 2.2 |
| C 17:0 | 0.6 | 0.5 | 0.4 |
| C 18:0 | 11.4 | 8.2 | 6.1 |
| C 18:1, cis, ω9 | 21.9 | 16.5 | 25.3 |
| C 18:1, trans, ω9 | 0.3 | 1.6 | 1.9 |
| C 18:2, ω 6 | 1.9 | 2.2 | 1.9 |
| C 18:3, ω 3, a | 0.6 | 0.4 | 0.6 |
| C 20:0 | 0.0 | 0.1 | 0.1 |
| C 20:1, ω9 | 0.1 | 0.1 | 0.2 |
| Saturated | 68.7 | 70.7 | 58.6 |
| Unsaturated | 27.8 | 23.1 | 33.3 |

AMF = anhydrous milk fat;
SCCO2 = super-critical carbon dioxide fraction (super olein).
MeltFrac = melt crystallization fraction separated at 10° C.

In some embodiments, the nutritional composition may include an enriched milk product, such as an enriched whey protein concentrate (eWPC). Enriched milk product generally refers to a milk product that has been enriched with certain milk fat globule membrane (MFGM) components, such as proteins and lipids found in the MFGM. The enriched milk product can be formed by, e.g., fractionation of non-human (e.g., bovine) milk. Enriched milk products have a total protein level which can range between 20% and 90%, more preferably between 68% and 80%, of which between 3% and 50% is MFGM proteins; in some embodiments, MFGM proteins make up from 7% to 13% of the enriched milk product protein content. Enriched milk products also comprise from 0.5% to 5% (and, at times, 1.2% to 2.8%) sialic acid, from 2% to 25% (and, in some embodiments, 4% to 10%) phospholipids, from 0.4% to 3% sphingomyelin, from 0.05% to 1.8%, and, in certain embodiments 0.10% to 0.3%, gangliosides and from 0.02% to about 1.2%, more preferably from 0.2% to 0.9%, cholesterol. Thus, enriched milk products include desirable components at levels higher than found in bovine and other non-human milks.

In some embodiments, the enriched milk product may contain certain polar lipids such as (1) Glycerophospholipids such as phosphatidylcholine (PC), phosphatidylethanolamine (PE), phosphatidylserine (PS), and phosphatidylinositol (PI), and their derivatives and (2) Sphingoids or sphingolipids such as sphingomyelin (SM) and glycosphingolipids comprising cerebrosides (neutral glycosphingolipids containing uncharged sugars) and the gangliosides (GG, acidic glycosphingolipids containing sialic acid) and their derivatives.

PE is a phospholipid found in biological membranes, particularly in nervous tissue such as the white matter of brain, nerves, neural tissue, and in spinal cord, where it makes up 45% of all phospholipids. Sphingomyelin is a type of sphingolipid found in animal cell membranes, especially in the membranous myelin sheath that surrounds some nerve cell axons. It usually consists of phosphocholine and ceramide, or a phosphoethanolamine head group; therefore, sphingomyelins can also be classified as sphingophospholipids. In humans, SM represents ˜85% of all sphingolipids, and typically makes up 10-20 mol % of plasma membrane lipids. Sphingomyelins are present in the plasma membranes of animal cells and are especially prominent in myelin, a membranous sheath that surrounds and insulates the axons of some neurons.

In some embodiments, the enriched milk product includes eWPC. The eWPC may be produced by any number of fractionation techniques. These techniques include but are not limited to melting point fractionation, organic solvent fractionation, super critical fluid fractionation, and any variants and combinations thereof. Alternatively, eWPC is available commercially, including under the trade names Lacprodan MFGM-10 and Lacprodan PL-20, both available from Arla Food Ingredients of Viby, Denmark. With the addition of eWPC, the lipid composition of infant formulas and other pediatric nutritional compositions can more closely resemble that of human milk. For instance, the theoretical values of phospholipids (mg/L) and gangliosides (mg/L) in an exemplary infant formula which includes Lacprodan MFGM-10 or Lacprodan PL-20 can be calculated as shown in Table 2:

TABLE 2

| | Total | | | | | | | |
|---|---|---|---|---|---|---|---|---|
| Item | milk PL | SM | PE | PC | PI | PS | Other PL | GD3 |
| MFGM-10 | 330 | 79.2 | 83.6 | 83.6 | 22 | 39.6 | 22 | 10.1 |
| PL-20 | 304 | 79 | 64 | 82 | 33 | 33 | 12.2 | 8.5 |

PL: phospholipids;
SM: sphingomyelin;
PE: phosphatidyl ethanolamine;
PC: phosphatidyl choline;
PI: phosphatidyl inositol;
PS: phosphatidyl serine;
GD3: ganglioside GD3.

In some embodiments, the eWPC is included in the nutritional composition of the present disclosure at a level of about 0.5 grams per liter (g/L) to about 10 g/L; in other embodiments, the eWPC is present at a level of about 1 g/L to about 9 g/L. In still other embodiments, eWPC is present in the nutritional composition at a level of about 3 g/L to about 8 g/L. Alternatively, in certain embodiments, the eWPC is included in the nutritional composition of the present disclosure at a level of about 0.06 grams per 100 Kcal (g/100 Kcal) to about 1.5 g/100 Kcal; in other embodiments, the eWPC is present at a level of about 0.3 g/100 Kcal to about 1.4 g/100 Kcal. In still other embodiments, the eWPC is present in the nutritional composition at a level of about 0.4 g/100 Kcal to about 1 g/100 Kcal.

Total phospholipids in the nutritional composition disclosed herein (i.e., including phospholipids from the eWPC as well as other components, but not including phospholipids from plant sources such as soy lecithin, if used) is in a range of about 50 mg/L to about 2000 mg/L; in some embodiments it is about 100 mg/L to about 1000 mg/L, or about 150 mg/L to about 550 mg/L. In certain embodiments, the eWPC component also contributes sphingomyelin in a range of about 10 mg/L to about 200 mg/L; in other embodiments, it is about 30 mg/L to about 150 mg/L, or about 50 mg/L to about 140 mg/L. And, the eWPC can also contribute gangliosides, which in some embodiments, are present in a range of about 2 mg/L to about 40 mg/L, or, in other embodiments about 6 mg/L to about 35 mg/L. In still other embodiments, the gangliosides are present in a range of about 9 mg/L to about 30 mg/L. In some embodiments, total phospholipids in the nutritional composition (again not including phospholipids from plant sources such as soy lecithin) is in a range of about 6 mg/100 Kcal to about 300 mg/100 Kcal; in some embodiments it is about 12 mg/100 Kcal to about 150 mg/100 Kcal, or about 18 mg/100 Kcal to about 85 mg/100 Kcal. In certain embodiments, the eWPC also contributes sphingomyelin in a range of about 1 mg/100 Kcal to about 30 mg/100 Kcal; in other embodiments, it is about 3.5 mg/100 Kcal to about 24 mg/100 Kcal, or about 6 mg/100 Kcal to about 21 mg/100 Kcal. And, gangliosides can be present in a range of about 0.25 mg/100 Kcal to about 6 mg/100 Kcal, or, in other embodiments about 0.7 mg/100 Kcal to about 5.2 mg/100 Kcal. In still other embodiments, the gangliosides are present in a range of about 1.1 mg/100 Kcal to about 4.5 mg/100 Kcal.

In some embodiments, the eWPC contains sialic acid (SA). Generally, the term sialic acid (SA) is used to generally refer to a family of derivatives of neuraminic acid. N-acetylneuraminic acid (Neu5Ac) and N-glycolylneuraminic acid (Neu5Gc) are among the most abundant naturally found forms of SA, especially Neu5Ac in human and cow's milk. Mammalian brain tissue contains the highest levels of SA because of its incorporation into brain-specific proteins such as neural cell adhesion molecule (NCAM) and lipids (e.g., gangliosides). It is considered that SA plays a role in neural development and function, learning, cognition, and memory throughout the life. In human milk, SA exists as free and bound forms with oligosaccharides, protein and lipid. The content of SA in human milk varies with lactation stage, with the highest level found in colostrum. However, most SA in bovine milk is bound with proteins, compared to the majority of SA in human milk bound to free oligosaccharides. Sialic acid can be incorporated in to the disclosed nutritional composition as is, or it can be provided by incorporating casein glycomacropeptide (cGMP) having enhanced sialic acid content, as discussed in U.S. Pat. Nos. 7,867,541 and 7,951,410, the disclosure of each of which are incorporated by reference herein.

When present, sialic acid can be incorporated into the nutritional composition of the present disclosure at a level of about 100 mg/L to about 800 mg/L, including both inherent sialic acid from the eWPC and exogenous sialic acid and sialic acid from sources such as cGMP. In some embodiments, sialic acid is present at a level of about 120 mg/L to about 600 mg/L; in other embodiments the level is about 140 mg/L to about 500 mg/L. In certain embodiments, sialic acid may be present in an amount from about 1 mg/100 Kcals to about 120 mg/100 Kcal. In other embodiments sialic acid may be present in an amount from about 14 mg/100 Kcal to about 90 mg/100 Kcal. In yet other embodiments, sialic acid may be present in an amount from about 15 mg/100 Kcal to about 75 mg/100 Kcal.

In certain embodiments, the nutritional composition may further include inositol. Without being bound by any particular theory, it has been found that nutritional supplementation of inositol represents a feasible and effective approach to promote oligodendrocyte survival and proliferation in a dose dependent manner, resulting in a consistent increase in the number of oligodendrocyte precursor cells. Accordingly, providing a nutritional composition having a combination of dietary butyrate and inositol may act synergistically to promote oligodendrocyte survival and proliferation of OPCs into oligodendric cells. Accordingly, nutritional supplementation with inositol provides benefits for enhanced developmental myelination by which it translates into a fundamental benefit for brain development. Given the importance of functional myelination, nutritional supplementation of inositol in combination with dietary butyrate is beneficial to pediatric subjects by enhancing brain development and health.

Furthermore, it is noted that the inclusion of dietary butyrate into nutritional compositions, such as pediatric compositions and infant formulas, may provide undesirable sensory characteristics, such as poor taste and smell. Indeed, dietary butyrate is generally not supplemented in effective levels given the negative organoleptic properties that result. However, the combination of inositol with dietary butyrate provide a nutritional composition with improved organoleptic properties, such as improved taste, because the sweet taste of inositol provides further advantages in terms of palatability to pediatric consumers. Thus, incorporating the combination of dietary and inositol into the nutritional composition provides a nutritional composition with improved organoleptic properties.

As such, in certain embodiments, inositol is present in the nutritional compositions of the present disclosure at a level of at least about 4 mg/100 Kcal; in other embodiments, inositol should be present at a level of no greater than about 70 mg/100 Kcal. In still other embodiments, the nutritional composition comprises inositol at a level of about 5 mg/100 Kcal to about 65 mg/100 Kcal. In a further embodiment, inositol is present in the nutritional composition at a level of about 7 mg/100 Kcal to about 50 mg/100 Kcal. Moreover, inositol can be present as exogenous inositol or inherent inositol. In embodiments, a major fraction of the inositol (i.e., at least 40%) is exogenous inositol. In certain embodiments, the ratio of exogenous to inherent inositol is at least 50:50; in other embodiments, the ratio of exogenous to inherent inositol is at least 60:40.

In certain embodiments, the nutritional composition may further include at least one organosulfur compound including, alpha-lipoic acid (ALA), allyl sulfide, allyl disulfide, sulforaphane (SFN), L-sulforaphane (L-SFN), and combinations thereof.

Allyl sulfide, also commonly known as diallyl sulfide is an organosulfur compound with the chemical formula C₆H₁₀S. Allyl sulfides, for example diallyl sulfide, diallyl disulfide, and diallyl trisulfide, are principle constituents of garlic oil. In vivo allyl sulfide may be converted to diallyl sulfoxide and diallyl sulfone by cytochrome P450 2E1 (CYP2E1).

Sulforaphane (SFN) is a molecule within the isothiocyanate group of organosulfur compounds having the molecular formula C₆H₁₁NOS₂. SFN and its isomers, for example L-Sulforaphane (“L-SFN”), are known to exhibit anti-cancer and antimicrobial properties in experimental models. SFN may be obtained from cruciferous vegetables, such as broccoli, Brussels sprouts or cabbage. SFN is produced when the enzyme myrosinase reacts with glucoraphanin, a glucosinolate, transforming glucoraphanin into SFN.

In some embodiments, the at least one organosulfur compound incorporated into the nutritional composition comprises ALA. Examples of ALA suitable for use in the nutritional composition disclosed herein include, but are not limited to, enantiomers and racemic mixtures of ALA, including, R-lipoic acid “RLA”, S-lipoic acid “SLA”, and R/S-LA. Also suitable is R-lipoic acid stabilized with either sodium (“Na-RALA”) or potassium as Potassium-R-Lipoate.

When incorporated into a nutritional composition for practicing the method of the present disclosure, ALA may be present in the nutritional composition, in some embodiments in an amount from about 0.1 mg/100 Kcals to about 35 mg/100 Kcals. In some embodiments, ALA may be present in an amount from about 2.0 mg/100 Kcals to about 25 mg/100 Kcals. In still other embodiments, ALA may be present in an amount from about 5.0 mg/100 Kcals to about 15 mg/100 Kcals.

In some embodiments, the organosulfur compound incorporated into the nutritional composition is allyl disulfide. Allyl disulfide may be present in the nutritional composition, in some embodiments, in an amount from about 1 mg/100 Kcals to about 170 mg/100 Kcals. In still some embodiments, allyl disulfide may be present from about 50 mg/100 Kcals to about 120 mg/100 Kcals. In still other embodiments, allyl disulfide may be present from about 75 mg/100 Kcals to about 100 mg/100 Kcals.

Sulforaphane, which includes L-sulforaphane, may be incorporated into the nutritional composition in an amount from about 1.5 mg/100 Kcals to about 7.5 mg/100 Kcals. Still in some embodiments, sulforaphane may be present in an amount from about 2 mg/100 Kcals to about 6 mg/100 Kcals. In some embodiments, sulforaphane may be present in an amount from about 3 mg/100 Kcals to about 5 mg/100 Kcals.

In some embodiments, the nutritional composition comprises a source of flavan-3-ols. Flavan-3-ols which are suitable for use in the inventive nutritional composition include catechin, epicatechin (EC), gallocatechin, epigallocatechin (EGC), epicatechin gallate (ECG), epicatechin-3-gallate, epigallocatechin gallate (EGCG), and combinations thereof. In certain embodiments, the nutritional composition comprises EGCG.

In some embodiments, EGCG may be present in the nutritional composition in an amount from about 0.01 mg/100 Kcal to about 18 mg/100 Kcal. In some embodiments, EGCG may be present in an amount of from about 0.06 mg/100 Kcal to about 10 mg/100 Kcal. In some embodiments, EGCG may be present in an amount of from about 0.10 mg/100 Kcal to about 5.0 mg/100 Kcal. In some embodiments, EGCG may be present in an amount of from about 0.90 mg/100 Kcal to about 3.0 mg/100 Kcal.

The nutritional composition of the present disclosure also includes at least one probiotic; in a preferred embodiment, the probiotic comprises Lactobacillus rhamnosus GG (“LGG”) (ATCC 53103). In certain other embodiments, the probiotic may be selected from any other Lactobacillus species, Bifidobacterium species, Bifidobacterium longum BB536 (BL999, ATCC: BAA-999), Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof.

The amount of the probiotic may vary from about 1×10⁴ to about 1.5×10¹² cfu of probiotic(s) per 100 Kcal. In some embodiments the amount of probiotic may be from about 1×10⁶ to about 1×10⁹ cfu of probiotic(s) per 100 Kcal. In certain other embodiments the amount of probiotic may vary from about 1×10⁷ cfu/100 Kcal to about 1×10⁸ cfu of probiotic(s) per 100 Kcal.

As noted, in a preferred embodiment, the probiotic comprises LGG. LGG is a probiotic strain isolated from healthy human intestinal flora. It was disclosed in U.S. Pat. No. 5,032,399 to Gorbach, et al., which is herein incorporated in its entirety, by reference thereto. LGG is resistant to most antibiotics, stable in the presence of acid and bile, and attaches avidly to mucosal cells of the human intestinal tract. It survives for 1-3 days in most individuals and up to 7 days in 30% of subjects. In addition to its colonization ability, LGG also beneficially affects mucosal immune responses. LGG is deposited with the depository authority American Type Culture Collection (“ATCC”) under accession number ATCC 53103.

In an embodiment, the probiotic(s) may be viable or non-viable. The probiotics useful in the present disclosure may be naturally-occurring, synthetic or developed through the genetic manipulation of organisms, whether such source is now known or later developed.

In some embodiments, the nutritional composition may include a source comprising probiotic cell equivalents, which refers to the level of non-viable, non-replicating probiotics equivalent to an equal number of viable cells. The term “non-replicating” is to be understood as the amount of non-replicating microorganisms obtained from the same amount of replicating bacteria (cfu/g), including inactivated probiotics, fragments of DNA, cell wall or cytoplasmic compounds. In other words, the quantity of non-living, non-replicating organisms is expressed in terms of cfu as if all the microorganisms were alive, regardless whether they are dead, non-replicating, inactivated, fragmented etc. In non-viable probiotics are included in the nutritional composition, the amount of the probiotic cell equivalents may vary from about 1×10⁴ to about 1.5×10¹⁰ cell equivalents of probiotic(s) per 100 Kcal. In some embodiments the amount of probiotic cell equivalents may be from about 1×10⁶ to about 1×10⁹ cell equivalents of probiotic(s) per 100 Kcal nutritional composition. In certain other embodiments the amount of probiotic cell equivalents may vary from about 1×10⁷ to about 1×10⁸ cell equivalents of probiotic(s) per 100 Kcal of nutritional composition.

In some embodiments, the probiotic source incorporated into the nutritional composition may comprise both viable colony-forming units, and non-viable cell-equivalents.

While, probiotics may be helpful in pediatric patients, the administration of viable bacteria to pediatric subjects, and particularly preterm infants, with impaired intestinal defenses and immature gut barrier function may not be feasible due to the risk of bacteremia. Therefore, there is a need for compositions that can provide the benefits of probiotics without introducing viable bacteria into the intestinal tract of pediatric subjects

While not wishing to be bound by theory, it is believed that a culture supernatant from batch cultivation of a probiotic, and in particular embodiments, LGG, provides beneficial gastrointestinal benefits. It is further believed that the beneficial effects on gut barrier function can be attributed to the mixture of components (including proteinaceous materials, and possibly including (exo)polysaccharide materials) that are released into the culture medium at a late stage of the exponential (or “log”) phase of batch cultivation of LGG. The composition will be hereinafter referred to as “culture supernatant.”

Accordingly, in some embodiments, the nutritional composition includes a culture supernatant from a late-exponential growth phase of a probiotic batch-cultivation process. Without wishing to be bound by theory, it is believed that the activity of the culture supernatant can be attributed to the mixture of components (including proteinaceous materials, and possibly including (exo)polysaccharide materials) as found released into the culture medium at a late stage of the exponential (or “log”) phase of batch cultivation of the probiotic. The term “culture supernatant” as used herein, includes the mixture of components found in the culture medium. The stages recognized in batch cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

In an embodiment, a culture supernatant is obtainable by a process comprising the steps of (a) subjecting a probiotic such as LGG to cultivation in a suitable culture medium using a batch process; (b) harvesting the culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removing liquid contents from the culture supernatant so as to obtain the composition.

The culture supernatant may comprise secreted materials that are harvested from a late exponential phase. The late exponential phase occurs in time after the mid exponential phase (which is halftime of the duration of the exponential phase, hence the reference to the late exponential phase as being the second half of the time between the lag phase and the stationary phase). In particular, the term “late exponential phase” is used herein with reference to the latter quarter portion of the time between the lag phase and the stationary phase of the LGG batch-cultivation process. In some embodiments, the culture supernatant is harvested at a point in time of 75% to 85% of the duration of the exponential phase, and may be harvested at about ⅚ of the time elapsed in the exponential phase.

The culture supernatant is believed to contain a mixture of amino acids, oligo- and polypeptides, and proteins, of various molecular weights. The composition is further believed to contain polysaccharide structures and/or nucleotides.

In some embodiments, the culture supernatant of the present disclosure excludes low molecular weight components, generally below 6 kDa, or even below 5 kDa. In these and other embodiments, the culture supernatant does not include lactic acid and/or lactate salts. These lower molecular weight components can be removed, for example, by filtration or column chromatography.

The culture supernatant of the present disclosure can be formulated in various ways for administration to pediatric subjects. For example, the culture supernatant can be used as such, e.g. incorporated into capsules for oral administration, or in a liquid nutritional composition such as a drink, or it can be processed before further use. Such processing generally involves separating the compounds from the generally liquid continuous phase of the supernatant. This preferably is done by a drying method, such as spray-drying or freeze-drying (lyophilization). Spray-drying is preferred. In a preferred embodiment of the spray-drying method, a carrier material will be added before spray-drying, e.g., maltodextrin DE29.

The LGG culture supernatant of the present disclosure, whether added in a separate dosage form or via a nutritional product, will generally be administered in an amount effective in promoting gut regeneration, promoting gut maturation and/or protecting gut barrier function. The effective amount is preferably equivalent to 1×10⁴ to about 1×10¹² cell equivalents of live probiotic bacteria per kg body weight per day, and more preferably 10⁸-10⁹ cell equivalents per kg body weight per day. In other embodiments, the amount of cell equivalents may vary from about 1×10⁴ to about 1.5×10¹⁰ cell equivalents of probiotic(s) per 100 Kcal. In some embodiments the amount of probiotic cell equivalents may be from about 1×10⁶ to about 1×10⁹ cell equivalents of probiotic(s) per 100 Kcal nutritional composition. In certain other embodiments the amount of probiotic cell equivalents may vary from about 1×10⁷ to about 1×10⁸ cell equivalents of probiotic(s) per 100 Kcal of nutritional composition.

Without being bound by any theory, the unique combination of nutrients in the disclosed nutritional composition(s) is believed to be capable of providing novel and unexpected benefits for infants and children. Moreover, the benefit of this nutritional composition is believed to be obtained during infancy, and also by including it as part of a diverse diet as the child continues to grow and develop.

In some embodiments, a soluble mediator preparation is prepared from the culture supernatant as described below. Furthermore, preparation of an LGG soluble mediator preparation is described in US 2013/0251829 and US 2011/0217402, each of which is incorporated by reference in its entirety.

In certain embodiments, the soluble mediator preparation is obtainable by a process comprising the steps of (a) subjecting a probiotic such as LGG to cultivation in a suitable culture medium using a batch process; (b) harvesting a culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removal of any remaining cells using 0.22 μm sterile filtration to provide the soluble mediator preparation; (e) removing liquid contents from the soluble mediator preparation so as to obtain the composition.

In certain embodiments, secreted materials are harvested from a late exponential phase. The late exponential phase occurs in time after the mid exponential phase (which is halftime of the duration of the exponential phase, hence the reference to the late exponential phase as being the second half of the time between the lag phase and the stationary phase). In particular, the term “late exponential phase” is used herein with reference to the latter quarter portion of the time between the lag phase and the stationary phase of the LGG batch-cultivation process. In a preferred embodiment of the present disclosure and embodiments thereof, harvesting of the culture supernatant is at a point in time of 75% to 85% of the duration of the exponential phase, and most preferably is at about ⅚ of the time elapsed in the exponential phase.

The term “cultivation” or “culturing” refers to the propagation of microorganisms, in this case LGG, on or in a suitable medium. Such a culture medium can be of a variety of kinds, and is particularly a liquid broth, as customary in the art. A preferred broth, e.g., is MRS broth as generally used for the cultivation of lactobacilli. MRS broth generally comprises polysorbate, acetate, magnesium and manganese, which are known to act as special growth factors for lactobacilli, as well as a rich nutrient base. A typical composition comprises (amounts in g/liter): peptone from casein 10.0; yeast extract 4.0; D(+)-glucose 20.0; dipotassium hydrogen phosphate 2.0; Tween® 80 1.0; triammonium citrate 2.0; sodium acetate 5.0; magnesium sulphate 0.2; manganese sulphate 0.04.

In certain embodiments, the soluble mediator preparation is incorporated into an infant formula or other nutritional composition. The harvesting of secreted bacterial products brings about a problem that the culture media cannot easily be deprived of undesired components. This specifically relates to nutritional products for relatively vulnerable subjects, such as infant formula or clinical nutrition. This problem is not incurred if specific components from a culture supernatant are first isolated, purified, and then applied in a nutritional product. However, it is desired to make use of a more complete culture supernatant. This would serve to provide a soluble mediator composition better reflecting the natural action of the probiotic (e.g. LGG).

Accordingly, it is desired to ensure that the composition harvested from LGG cultivation does not contain components (as may present in the culture medium) that are not desired, or generally accepted, in such formula. With reference to polysorbate regularly present in MRS broth, media for the culturing of bacteria may include an emulsifying non-ionic surfactant, e.g. on the basis of polyethoxylated sorbitan and oleic acid (typically available as Tween® polysorbates, such as Tween® 80). Whilst these surfactants are frequently found in food products, e.g. ice cream, and are generally recognized as safe, they are not in all jurisdictions considered desirable, or even acceptable for use in nutritional products for relatively vulnerable subjects, such as infant formula or clinical nutrition.

Therefore, in some embodiments, a preferred culture medium of the disclosure is devoid of polysorbates such as Tween 80. In a preferred embodiment of the disclosure and/or embodiments thereof the culture medium may comprise an oily ingredient selected from the group consisting of oleic acid, linseed oil, olive oil, rape seed oil, sunflower oil and mixtures thereof. It will be understood that the full benefit of the oily ingredient is attained if the presence of a polysorbate surfactant is essentially or entirely avoided.

More particularly, in certain embodiments, an MRS medium is devoid of polysorbates. Also preferably medium comprises, in addition to one or more of the foregoing oils, peptone (typically 0-10 g/L, especially 0.1-10 g/L), yeast extract (typically 4-50 g/L), D(+) glucose (typically 20-70 g/L), dipotassium hydrogen phosphate (typically 2-4 g/L), sodium acetate trihydrate (typically 4-5 g/L), triammonium citrate (typically 2-4 g/L), magnesium sulfphate heptahydrate (typically 0.2-0.4 g/L) and/or manganous sulphate tetrahydrate (typically 0.05-0.08 g/L).

The culturing is generally performed at a temperature of 20° C. to 45° C., more particularly at 35° C. to 40° C., and more particularly at 37° C. In some embodiments, the culture has a neutral pH, such as a pH of between pH 5 and pH 7, preferably pH 6.

In some embodiments, the time point during cultivation for harvesting the culture supernatant, i.e., in the aforementioned late exponential phase, can be determined, e.g. based on the OD600 nm and glucose concentration. OD600 refers to the optical density at 600 nm, which is a known density measurement that directly correlates with the bacterial concentration in the culture medium.

The culture supernatant can be harvested by any known technique for the separation of culture supernatant from a bacterial culture. Such techniques are known in the art and include, e.g., centrifugation, filtration, sedimentation, and the like. In some embodiments, LGG cells are removed from the culture supernatant using 0.22 μm sterile filtration in order to produce the soluble mediator preparation. The probiotic soluble mediator preparation thus obtained may be used immediately, or be stored for future use. In the latter case, the probiotic soluble mediator preparation will generally be refrigerated, frozen or lyophilized. The probiotic soluble mediator preparation may be concentrated or diluted, as desired.

The soluble mediator preparation is believed to contain a mixture of amino acids, oligo- and polypeptides, and proteins, of various molecular weights. The composition is further believed to contain polysaccharide structures and/or nucleotides.

In some embodiments, the soluble mediator preparation of the present disclosure excludes lower molecular weight components, generally below 6 kDa, or even below 5 kDa. In these and other embodiments, the soluble mediator preparation does not include lactic acid and/or lactate salts. These lower molecular weight components can be removed, for example, by filtration or column chromatography. In some embodiments, the culture supernatant is subjected to ultrafiltration with a 5 kDa membrane in order to retain constituents over 5 kDa. In other embodiments, the culture supernatant is desalted using column chromatography to retain constituents over 6 kDa.

The soluble mediator preparation of the present disclosure can be formulated in various ways for administration to pediatric subjects. For example, the soluble mediator preparation can be used as such, e.g. incorporated into capsules for oral administration, or in a liquid nutritional composition such as a drink, or it can be processed before further use. Such processing generally involves separating the compounds from the generally liquid continuous phase of the supernatant. This preferably is done by a drying method, such as spray-drying or freeze-drying (lyophilization). In a preferred embodiment of the spray-drying method, a carrier material will be added before spray-drying, e.g., maltodextrin DE29.

Probiotic bacteria soluble mediator preparations, such as the LGG soluble mediator preparation disclosed herein, advantageously possess gut barrier enhancing activity by promoting gut barrier regeneration, gut barrier maturation and/or adaptation, gut barrier resistance and/or gut barrier function. The present LGG soluble mediator preparation may accordingly be particularly useful in treating subjects, particularly pediatric subjects, with impaired gut barrier function, such as short bowel syndrome or NEC. The soluble mediator preparation may be particularly useful for infants and premature infants having impaired gut barrier function and/or short bowel syndrome.

Probiotic bacteria soluble mediator preparation, such as the LGG soluble mediator preparation of the present disclosure, also advantageously reduce visceral pain sensitivity in subjects, particularly pediatric subjects experiencing gastrointestinal pain, food intolerance, allergic or non-allergic inflammation, colic, IBS, and infections.

In an embodiment, the nutritional composition may include prebiotics. In certain embodiments, the nutritional composition includes prebiotics that may stimulate endogenous butyrate production. For example, in some embodiments the component for stimulating endogenous butyrate production comprises a microbiota-stimulating component that is a prebiotic including both polydextrose (“PDX”) and galacto-oligosaccharides (“GOS”). A prebiotic component including PDX and GOS can enhance butyrate production by microbiota.

In addition to PDX and GOS, the nutritional composition may also contain one or more other prebiotics which can exert additional health benefits, which may include, but are not limited to, selective stimulation of the growth and/or activity of one or a limited number of beneficial gut bacteria, stimulation of the growth and/or activity of ingested probiotic microorganisms, selective reduction in gut pathogens, and favorable influence on gut short chain fatty acid profile. Such prebiotics may be naturally-occurring, synthetic, or developed through the genetic manipulation of organisms and/or plants, whether such new source is now known or developed later. Prebiotics useful in the present disclosure may include oligosaccharides, polysaccharides, and other prebiotics that contain fructose, xylose, soya, galactose, glucose and mannose.

More specifically, prebiotics useful in the present disclosure include PDX and GOS, and can, in some embodiments, also include, PDX powder, lactulose, lactosucrose, raffinose, gluco-oligosaccharide, inulin, fructo-oligosaccharide (FOS), isomalto-oligosaccharide, soybean oligosaccharides, lactosucrose, xylo-oligosaccharide (XOS), chito-oligosaccharide, manno-oligosaccharide, aribino-oligosaccharide, siallyl-oligosaccharide, fuco-oligosaccharide, and gentio-oligosaccharides.

In an embodiment, the total amount of prebiotics present in the nutritional composition may be from about 1.0 g/L to about 10.0 g/L of the composition. More preferably, the total amount of prebiotics present in the nutritional composition may be from about 2.0 g/L and about 8.0 g/L of the composition. In some embodiments, the total amount of prebiotics present in the nutritional composition may be from about 0.01 g/100 Kcal to about 1.5 g/100 Kcal. In certain embodiments, the total amount of prebiotics present in the nutritional composition may be from about 0.15 g/100 Kcal to about 1.5 g/100 Kcal. In some embodiments, the prebiotic component comprises at least 20% w/w PDX and GOS.

The amount of PDX in the nutritional composition may, in an embodiment, be within the range of from about 0.015 g/100 Kcal to about 1.5 g/100 Kcal. In another embodiment, the amount of polydextrose is within the range of from about 0.2 g/100 Kcal to about 0.6 g/100 Kcal. In some embodiments, PDX may be included in the nutritional composition in an amount sufficient to provide between about 1.0 g/L and 10.0 g/L. In another embodiment, the nutritional composition contains an amount of PDX that is between about 2.0 g/L and 8.0 g/L. And in still other embodiments, the amount of PDX in the nutritional composition may be from about 0.05 g/100 Kcal to about 1.5 g/100 Kcal.

The prebiotic component also comprises GOS. The amount of GOS in the nutritional composition may, in an embodiment, be from about 0.015 g/100 Kcal to about 1.0 g/100 Kcal. In another embodiment, the amount of GOS in the nutritional composition may be from about 0.2 g/100 Kcal to about 0.5 g/100 Kcal.

In a particular embodiment, GOS and PDX are supplemented into the nutritional composition in a total amount of at least about 0.015 g/100 Kcal or about 0.015 g/100 Kcal to about 1.5 g/100 Kcal. In some embodiments, the nutritional composition may comprise GOS and PDX in a total amount of from about 0.1 to about 1.0 g/100 Kcal.

In some embodiments, the nutritional composition includes a protein equivalent source, wherein the protein equivalent source includes a peptide component comprising SEQ ID NO 4, SEQ ID NO 13, SEQ ID NO 17, SEQ ID NO 21, SEQ ID NO 24, SEQ ID NO 30, SEQ ID NO 31, SEQ ID NO 32, SEQ ID NO 51, SEQ ID NO 57, SEQ ID NO 60, and SEQ ID NO 63. In some embodiments, the peptide component may comprise additional peptides disclosed in Table 3. For example, the composition may include at least 10 additional peptides disclosed in Table 3. In some embodiments, 20% to 80% of the protein equivalent source comprises the peptide component, and 20% to 80% of the protein equivalent source comprises an intact protein, a partially hydrolyzed protein, and combinations thereof. In some embodiments, the term additional means selecting different peptides than those enumerated.

In another embodiment, 1% to about 99% of the protein equivalent source includes a peptide component comprising at least 3 peptides selected from the group consisting of SEQ ID NO 4, SEQ ID NO 13, SEQ ID NO 17, SEQ ID NO 21, SEQ ID NO 24, SEQ ID NO 30, SEQ ID NO 31, SEQ ID NO 32, SEQ ID NO 51, SEQ ID NO 57, SEQ ID NO 60, and SEQ ID NO 63, and at least 5 additional peptides selected from Table 3; and wherein 1% to 99% of the protein equivalent source comprises an intact protein, a partially hydrolyzed protein, or combinations thereof. In some embodiments, 20% to 80% of the protein equivalent source includes a peptide component comprising at least 3 peptides selected from the group consisting of SEQ ID NO 4, SEQ ID NO 13, SEQ ID NO 17, SEQ ID NO 21, SEQ ID NO 24, SEQ ID NO 30, SEQ ID NO 31, SEQ ID NO 32, SEQ ID NO 51, SEQ ID NO 57, SEQ ID NO 60, and SEQ ID NO 63, and at least 5 additional peptides selected from Table 3; and wherein 20% to 80% of the protein equivalent source comprises an intact protein, a partially hydrolyzed protein, or combinations thereof.

Table 3 below identifies the amino acid sequences of the peptides that may be included in the peptide component of the present nutritional compositions.

TABLE 3

| Seq. | | | | | | | | | | | | |
|---|---|---|---|---|---|---|---|---|---|---|---|---|
| ID | Amino Acid Sequence | | | | | | | | | | | (aa) |
| 1 | Ala | Ile | Asn | Pro | Ser | Lys | Glu | Asn | | | | 8 |
| 2 | Ala | Pro | Phe | Pro | Glu | | | | | | | 5 |
| 3 | Asp | Ile | Gly | Ser | Glu | Ser | | | | | | 6 |
| 4 | Asp | Lys | Thr | Glu | Ile | Pro | Thr | | | | | 7 |
| 5 | Asp | Met | Glu | Ser | Thr | | | | | | | 5 |
| 6 | Asp | Met | Pro | Ile | | | | | | | | 4 |
| 7 | Asp | Val | Pro | Ser | | | | | | | | 4 |
| n/a | Glu | Asp | Ile | | | | | | | | | 3 |
| n/a | Glu | Leu | Phe | | | | | | | | | 3 |
| n/a | Glu | Met | Pro | | | | | | | | | 3 |
| 8 | Glu | Thr | Ala | Pro | Val | Pro | Leu | | | | | 7 |
| 9 | Phe | Pro | Gly | Pro | Ile | Pro | | | | | | 6 |
| 10 | Phe | Pro | Gly | Pro | Ile | Pro | Asn | | | | | 7 |
| 11 | Gly | Pro | Phe | Pro | | | | | | | | 4 |
| 12 | Gly | Pro | Ile | Val | | | | | | | | 4 |
| 13 | Ile | Gly | Ser | Glu | Ser | Thr | Glu | Asp | Gln | | | 9 |
| 14 | Ile | Gly | Ser | Ser | Ser | Glu | Glu | Ser | | | | 8 |
| 15 | Ile | Gly | Ser | Ser | Ser | Glu | Glu | Ser | Ala | | | 9 |
| 16 | Ile | Asn | Pro | Ser | Lys | Glu | | | | | | 6 |
| 17 | Ile | Pro | Asn | Pro | Ile | | | | | | | 5 |
| 18 | Ile | Pro | Asn | Pro | Ile | Gly | | | | | | 6 |
| 19 | Ile | Pro | Pro | Leu | Thr | Gln | Thr | Pro | Val | | | 9 |
| 20 | Ile | Thr | Ala | Pro | | | | | | | | 4 |
| 21 | Ile | Val | Pro | Asn | | | | | | | | 4 |
| 22 | Lys | His | Gln | Gly | Leu | Pro | Gln | | | | | 7 |
| 23 | Leu | Asp | Val | Thr | Pro | | | | | | | 5 |
| 24 | Leu | Glu | Asp | Ser | Pro | Glu | | | | | | 6 |
| 25 | Leu | Pro | Leu | Pro | Leu | | | | | | | 5 |
| 26 | Met | Glu | Ser | Thr | Glu | Val | | | | | | 6 |
| 27 | Met | His | Gln | Pro | His | Gln | Pro | Leu | Pro | Pro | Thr | 11 |
| 28 | Asn | Ala | Val | Pro | Ile | | | | | | | 5 |
| 29 | Asn | Glu | Val | Glu | Ala | | | | | | | 5 |
| n/a | Asn | Leu | Leu | | | | | | | | | 3 |
| 30 | Asn | Gln | Glu | Gln | Pro | Ile | | | | | | 6 |
| 31 | Asn | Val | Pro | Gly | Glu | | | | | | | 5 |
| 32 | Pro | Phe | Pro | Gly | Pro | Ile | | | | | | 6 |
| 33 | Pro | Gly | Pro | Ile | Pro | Asn | | | | | | 6 |
| 34 | Pro | His | Gln | Pro | Leu | Pro | Pro | Thr | | | | 8 |
| 35 | Pro | Ile | Thr | Pro | Thr | | | | | | | 5 |
| 36 | Pro | Asn | Pro | Ile | | | | | | | | 4 |
| 37 | Pro | Asn | Ser | Leu | Pro | Gln | | | | | | 6 |
| 38 | Pro | Gln | Leu | Glu | Ile | Val | Pro | Asn | | | | 8 |
| 39 | Pro | Gln | Asn | Ile | Pro | Pro | Leu | | | | | 7 |
| 40 | Pro | Val | Leu | Gly | Pro | Val | | | | | | 6 |
| 41 | Pro | Val | Pro | Gln | | | | | | | | 4 |
| 42 | Pro | Val | Val | Val | Pro | | | | | | | 5 |
| 43 | Pro | Val | Val | Val | Pro | Pro | | | | | | 6 |
| 44 | Ser | Ile | Gly | Ser | Ser | Ser | Glu | Glu | Ser | Ala | Glu | 11 |
| 45 | Ser | Ile | Ser | Ser | Ser | Glu | Glu | | | | | 7 |
| 46 | Ser | Ile | Ser | Ser | Ser | Glu | Glu | Ile | Val | Pro | Asn | 11 |
| 47 | Ser | Lys | Asp | Ile | Gly | Ser | Glu | | | | | 7 |
| 48 | Ser | Pro | Pro | Glu | Ile | Asn | | | | | | 6 |
| 49 | Ser | Pro | Pro | Glu | Ile | Asn | Thr | | | | | 7 |
| 50 | Thr | Asp | Ala | Pro | Ser | Phe | Ser | | | | | 7 |
| 51 | Thr | Glu | Asp | Glu | Leu | | | | | | | 5 |
| 52 | Val | Ala | Thr | Glu | Glu | Val | | | | | | 6 |
| 53 | Val | Leu | Pro | Val | Pro | | | | | | | 5 |
| 54 | Val | Pro | Gly | Glu | | | | | | | | 4 |
| 55 | Val | Pro | Gly | Glu | Ile | Val | | | | | | 6 |
| 56 | Val | Pro | Ile | Thr | Pro | Thr | | | | | | 6 |
| 57 | Val | Pro | Ser | Glu | | | | | | | | 4 |
| 58 | Val | Val | Pro | Pro | Phe | Leu | Gln | Pro | Glu | | | 9 |
| 59 | Val | Val | Val | Pro | Pro | | | | | | | 5 |
| 60 | Tyr | Pro | Phe | Pro | Gly | Pro | | | | | | 6 |
| 61 | Tyr | Pro | Phe | Pro | Gly | Pro | Ile | Pro | | | | 8 |
| 62 | Tyr | Pro | Phe | Pro | Gly | Pro | Ile | Pro | Asn | | | 9 |
| 63 | Tyr | Pro | Ser | Gly | Ala | | | | | | | 5 |
| 64 | Tyr | Pro | Val | Glu | Pro | | | | | | | 5 |

Table 4 below further identifies a subset of amino acid sequences from Table 3 that may be included in the peptide component disclosed herein.

TABLE 4

| Seq ID | | | | | | | | | | |
|---|---|---|---|---|---|---|---|---|---|---|
| Number | Amino Acid Sequence | | | | | | | | | (aa) |
| 4 | Asp | Lys | Thr | Glu | Ile | Pro | Thr | | | 7 |
| 13 | Ile | Gly | Ser | Glu | Ser | Thr | Glu | Asp | Gln | 9 |
| 17 | Ile | Pro | Asn | Pro | Ile | Gly | | | | 6 |
| 21 | Ile | Val | Pro | Asn | | | | | | 4 |
| 24 | Leu | Glu | Asp | Ser | Pro | Glu | | | | 6 |
| 30 | Asn | Gln | Glu | Gln | Pro | Ile | | | | 6 |
| 31 | Asn | Val | Pro | Gly | Glu | | | | | 5 |
| 32 | Pro | Phe | Pro | Gly | Pro | Ile | | | | 6 |
| 51 | Thr | Glu | Asp | Glu | Leu | | | | | 5 |
| 57 | Val | Pro | Ser | Glu | | | | | | 4 |
| 60 | Tyr | Pro | Phe | Pro | Gly | Pro | | | | 6 |
| 63 | Tyr | Pro | Ser | Gly | Ala | | | | | 5 |

In some embodiments, the peptide component may be present in the nutritional composition in an amount from about 0.2 g/100 Kcal to about 5.6 g/100 Kcal. In other embodiments the peptide component may be present in the nutritional composition in an amount from about 1 g/100 Kcal to about 4 g/100 Kcal. In still other embodiments, the peptide component may be present in the nutritional composition in an amount from about 2 g/100 Kcal to about 3 g/100 Kcal.

The peptide component disclosed herein may be formulated with other ingredients in the nutritional composition to provide appropriate nutrient levels for the target subject. In some embodiments, the peptide component is included in a nutritionally complete formula that is suitable to support normal growth.

The peptide component may be provided as an element of a protein equivalent source. In some embodiments, the peptides identified in Tables 3 and 4, may be provided by a protein equivalent source obtained from cow's milk proteins, including but not limited to bovine casein and bovine whey. In some embodiments, the protein equivalent source comprises hydrolyzed bovine casein or hydrolyzed bovine whey. Accordingly, in some embodiments, the peptides identified in Table 3 and Table 4 may be provided by a casein hydrolysate. Such peptides may be obtained by hydrolysis or may be synthesized in vitro by methods know to the skilled person.

A non-limiting example of a method of hydrolysis is disclosed herein. In some embodiments, this method may be used to obtain the protein hydrolysate and peptides of the present disclosure. The proteins are hydrolyzed using a proteolytic enzyme, Protease N. Protease N “Amano” is commercially available from Amano Enzyme U.S.A. Co., Ltd., Elgin, Ill. Protease N is a proteolytic enzyme preparation that is derived from the bacterial species Bacillus subtilis. The protease powder is specified as “not less than 150,000 units/g”, meaning that one unit of Protease N is the amount of enzyme which produces an amino acid equivalent to 100 micrograms of tyrosine for 60 minutes at a pH of 7.0. To produce the infant formula of the present disclosure, Protease N can be used at levels of about 0.5% to about 1.0% by weight of the total protein being hydrolyzed.

The protein hydrolysis by Protease N is typically conducted at a temperature of about 50° C. to about 60° C. The hydrolysis occurs for a period of time so as to obtain a degree of hydrolysis between about 4% and 10%. In a particular embodiment, hydrolysis occurs for a period of time so as to obtain a degree of hydrolysis between about 6% and 9%. In another embodiment, hydrolysis occurs for a period of time so as to obtain a degree of hydrolysis of about 7.5%. This level of hydrolysis may take between about one half hour to about 3 hours.

A constant pH should be maintained during hydrolysis. In the method of the present disclosure, the pH is adjusted to and maintained between about 6.5 and 8. In a particular embodiment, the pH is maintained at about 7.0.

In order to maintain the optimal pH of the solution of whey protein, casein, water and Protease N, a caustic solution of sodium hydroxide and/or potassium hydroxide can be used to adjust the pH during hydrolysis. If sodium hydroxide is used to adjust the pH, the amount of sodium hydroxide added to the solution should be controlled to the level that it comprises less than about 0.3% of the total solid in the finished protein hydrolysate. A 10% potassium hydroxide solution can also be used to adjust the pH of the solution to the desired value, either before the enzyme is added or during the hydrolysis process in order to maintain the optimal pH.

The amount of caustic solution added to the solution during the protein hydrolysis can be controlled by a pH-stat or by adding the caustic solution continuously and proportionally. The hydrolysate can be manufactured by standard batch processes or by continuous processes.

To better ensure the consistent quality of the protein partial hydrolysate, the hydrolysate is subjected to enzyme deactivation to end the hydrolysis process. The enzyme deactivation step may consist include at heat treatment at a temperature of about 82° C. for about 10 minutes. Alternatively, the enzyme can be deactivated by heating the solution to a temperature of about 92° C. for about 5 seconds. After enzyme deactivation is complete, the hydrolysate can be stored in a liquid state at a temperature lower than 10° C.

In some embodiments, the protein equivalent source comprises a hydrolyzed protein, which includes partially hydrolyzed protein and extensively hydrolyzed protein, such as casein. In some embodiments, the protein equivalent source comprises a hydrolyzed protein including peptides having a molar mass distribution of greater than 500 Daltons. In some embodiments, the hydrolyzed protein comprises peptides having a molar mass distribution in the range of from about 500 Daltons to about 1,500 Daltons. Still, in some embodiments the hydrolyzed protein may comprise peptides having a molar mass distribution range of from about 500 Daltons to about 2,000 Daltons.

In some embodiments, the protein equivalent source may comprise the peptide component, intact protein, hydrolyzed protein, including partially hydrolyzed protein and/or extensively hydrolyzed protein, and combinations thereof. In some embodiments, 1% to 99% of the protein equivalent source comprises the peptide component disclosed herein. In some embodiments, 10% to 90% of the protein equivalent source comprises the peptide component disclosed herein. In some embodiments, 20% to 80% of the protein equivalent source comprises the peptide component disclosed herein. In some embodiments, 30% to 60% of the protein equivalent source comprises the peptide component disclosed herein. In still other embodiments, 40% to 50% of the protein equivalent source comprises the peptide component.

In some embodiments, 1% to 99% of the protein equivalent source comprises intact protein, partially hydrolyzed protein, extensively hydrolyzed protein, or combinations thereof. In some embodiments, 10% to 90% of the protein equivalent source comprises intact protein, partially hydrolyzed protein, extensively hydrolyzed protein, or combinations thereof. In some embodiments, 20% to 80% of the protein equivalent source comprises intact protein, partially hydrolyzed protein, extensively hydrolyzed protein, or combinations thereof. In some embodiments, 40% to 70% of the protein equivalent source comprises intact proteins, partially hydrolyzed proteins, extensively hydrolyzed protein, or a combination thereof. In still further embodiments, 50% to 60% of the protein equivalent source may comprise intact proteins, partially hydrolyzed protein, extensively hydrolyzed protein, or a combination thereof.

In some embodiments the protein equivalent source comprises partially hydrolyzed protein having a degree of hydrolysis of less than 40%. In still other embodiments, the protein equivalent source may comprise partially hydrolyzed protein having a degree of hydrolysis of less than 25%, or less than 15%.

In some embodiments, the nutritional composition comprises between about 1 g and about 7 g of a protein equivalent source per 100 Kcal. In other embodiments, the nutritional composition comprises between about 3.5 g and about 4.5 g of protein equivalent source per 100 Kcal.

The nutritional composition(s) of the disclosure may also comprise a protein source. The protein source can be any used in the art, e.g., nonfat milk, whey protein, casein, soy protein, hydrolyzed protein, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate) and any combinations thereof.

In one embodiment, the proteins of the nutritional composition are provided as intact proteins. In other embodiments, the proteins are provided as a combination of both intact proteins and partially hydrolyzed proteins, with a degree of hydrolysis of between about 4% and 10%. In certain other embodiments, the proteins are more completely hydrolyzed. In still other embodiments, the protein source comprises amino acids. In yet another embodiment, the protein source may be supplemented with glutamine-containing peptides.

In a particular embodiment of the nutritional composition, the whey:casein ratio of the protein source is similar to that found in human breast milk. In an embodiment, the protein source comprises from about 40% to about 80% whey protein and from about 20% to about 60% casein.

In some embodiments the protein source may include a combination of milk powders and whey protein powders. In some embodiments, the protein source comprise from about 5 wt % to about 30% of nonfat milk powder based on the total weight of the nutritional composition and about 2 wt % to about 20 wt % of whey protein concentrate based on the total weight of the nutritional composition. Still in certain embodiments, the protein source comprise from about 10 wt % to about 20% of nonfat milk powder based on the total weight of the nutritional composition and about 5 wt % to about 15 wt % of whey protein concentrate based on the total weight of the nutritional composition.

In some embodiments, the nutritional composition comprises between about 1 g and about 7 g of a protein source per 100 Kcal. In other embodiments, the nutritional composition comprises between about 3.5 g and about 4.5 g of protein per 100 Kcal.

The nutritional composition(s) of the present disclosure may also comprise a carbohydrate source. Carbohydrate sources can be any used in the art, e.g., lactose, glucose, fructose, corn syrup solids, maltodextrins, sucrose, starch, rice syrup solids, and the like. The amount of carbohydrate in the nutritional composition typically can vary from between about 5 g and about 25 g/100 Kcal. In some embodiments, the amount of carbohydrate is between about 6 g and about 22 g/100 Kcal. In other embodiments, the amount of carbohydrate is between about 12 g and about 14 g/100 Kcal. In some embodiments, corn syrup solids are preferred. Moreover, hydrolyzed, partially hydrolyzed, and/or extensively hydrolyzed carbohydrates may be desirable for inclusion in the nutritional composition due to their easy digestibility. Specifically, hydrolyzed carbohydrates are less likely to contain allergenic epitopes.

Non-limiting examples of carbohydrate materials suitable for use herein include hydrolyzed or intact, naturally or chemically modified, starches sourced from corn, tapioca, rice or potato, in waxy or non-waxy forms. Non-limiting examples of suitable carbohydrates include various hydrolyzed starches characterized as hydrolyzed cornstarch, maltodextrin, maltose, corn syrup, dextrose, corn syrup solids, glucose, and various other glucose polymers and combinations thereof. Non-limiting examples of other suitable carbohydrates include those often referred to as sucrose, lactose, fructose, high fructose corn syrup, indigestible oligosaccharides such as fructooligosaccharides and combinations thereof.

In some embodiments, the nutritional composition described herein comprises a fat source. The enriched lipid fraction described herein may be the sole fat source or may be used in combination with any other suitable fat or lipid source for the nutritional composition as known in the art. In certain embodiments, appropriate fat sources include, but are not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

In some embodiment the nutritional composition comprises between about 1 g/100 Kcal to about 10 g/100 Kcal of a fat or lipid source. In some embodiments, the nutritional composition comprises between about 2 g/100 Kcal to about 7 g/100 Kcal of a fat source. In other embodiments the fat source may be present in an amount from about 2.5 g/100 Kcal to about 6 g/100 Kcal. In still other embodiments, the fat source may be present in the nutritional composition in an amount from about 3 g/100 Kcal to about 4 g/100 Kcal.

In some embodiments, the fat or lipid source comprises from about 10% to about 35% palm oil per the total amount of fat or lipid. In some embodiments, the fat or lipid source comprises from about 15% to about 30% palm oil per the total amount of fat or lipid. Yet in other embodiments, the fat or lipid source may comprise from about 18% to about 25% palm oil per the total amount of fat or lipid.

In certain embodiments, the fat or lipid source may be formulated to include from about 2% to about 16% soybean oil based on the total amount of fat or lipid. In some embodiments, the fat or lipid source may be formulated to include from about 4% to about 12% soybean oil based on the total amount of fat or lipid. In some embodiments, the fat or lipid source may be formulated to include from about 6% to about 10% soybean oil based on the total amount of fat or lipid.

In certain embodiments, the fat or lipid source may be formulated to include from about 2% to about 16% coconut oil based on the total amount of fat or lipid. In some embodiments, the fat or lipid source may be formulated to include from about 4% to about 12% coconut oil based on the total amount of fat or lipid. In some embodiments, the fat or lipid source may be formulated to include from about 6% to about 10% coconut oil based on the total amount of fat or lipid.

In certain embodiments, the fat or lipid source may be formulated to include from about 2% to about 16% sunflower oil based on the total amount of fat or lipid. In some embodiments, the fat or lipid source may be formulated to include from about 4% to about 12% sunflower oil based on the total amount of fat or lipid. In some embodiments, the fat or lipid source may be formulated to include from about 6% to about 10% sunflower oil based on the total amount of fat or lipid.

In some embodiments, the oils, i.e. sunflower oil, soybean oil, sunflower oil, palm oil, etc. are meant to cover fortified versions of such oils known in the art. For example, in certain embodiments, the use of sunflower oil may include high oleic sunflower oil. In other examples, the use of such oils may be fortified with certain fatty acids, as known in the art, and may be used in the fat or lipid source disclosed herein.

In some embodiments the nutritional composition may also include a source of LCPUFAs. In one embodiment the amount of LCPUFA in the nutritional composition is advantageously at least about 5 mg/100 Kcal, and may vary from about 5 mg/100 Kcal to about 100 mg/100 Kcal, more preferably from about 10 mg/100 Kcal to about 50 mg/100 Kcal. Non-limiting examples of LCPUFAs include, but are not limited to, DHA, ARA, linoleic (18:2 n-6), γ-linolenic (18:3 n-6), dihomo-γ-linolenic (20:3 n-6) acids in the n-6 pathway, α-linolenic (18:3 n-3), stearidonic (18:4 n-3), eicosatetraenoic (20:4 n-3), eicosapentaenoic (20:5 n-3), and docosapentaenoic (22:6 n-3).

In some embodiments, the LCPUFA included in the nutritional composition may comprise DHA. In one embodiment the amount of DHA in the nutritional composition is advantageously at least about 17 mg/100 Kcal, and may vary from about 5 mg/100 Kcal to about 75 mg/100 Kcal, more preferably from about 10 mg/100 Kcal to about 50 mg/100 Kcal.

In another embodiment, especially if the nutritional composition is an infant formula, the nutritional composition is supplemented with both DHA and ARA. In this embodiment, the weight ratio of ARA:DHA may be between about 1:3 and about 9:1. In a particular embodiment, the ratio of ARA:DHA is from about 1:2 to about 4:1.

The DHA and ARA can be in natural form, provided that the remainder of the LCPUFA source does not result in any substantial deleterious effect on the infant. Alternatively, the DHA and ARA can be used in refined form.

The disclosed nutritional composition described herein can, in some embodiments, also comprise a source of β-glucan. Glucans are polysaccharides, specifically polymers of glucose, which are naturally occurring and may be found in cell walls of bacteria, yeast, fungi, and plants. Beta glucans (β-glucans) are themselves a diverse subset of glucose polymers, which are made up of chains of glucose monomers linked together via beta-type glycosidic bonds to form complex carbohydrates.

β-1,3-glucans are carbohydrate polymers purified from, for example, yeast, mushroom, bacteria, algae, or cereals. The chemical structure of β-1,3-glucan depends on the source of the β-1,3-glucan. Moreover, various physiochemical parameters, such as solubility, primary structure, molecular weight, and branching, play a role in biological activities of β-1,3-glucans.

β-1,3-glucans are naturally occurring polysaccharides, with or without β-1,6-glucose side chains that are found in the cell walls of a variety of plants, yeasts, fungi and bacteria. β-1,3;1,6-glucans are those containing glucose units with (1,3) links having side chains attached at the (1,6) position(s). β-1,3;1,6 glucans are a heterogeneous group of glucose polymers that share structural commonalities, including a backbone of straight chain glucose units linked by a β-1,3 bond with β-1,6-linked glucose branches extending from this backbone. While this is the basic structure for the presently described class of β-glucans, some variations may exist. For example, certain yeast β-glucans have additional regions of β(1,3) branching extending from the β(1,6) branches, which add further complexity to their respective structures.

β-glucans derived from baker's yeast, Saccharomyces cerevisiae, are made up of chains of D-glucose molecules connected at the 1 and 3 positions, having side chains of glucose attached at the 1 and 6 positions. Yeast-derived β-glucan is an insoluble, fiber-like, complex sugar having the general structure of a linear chain of glucose units with a β-1,3 backbone interspersed with β-1,6 side chains that are generally 6-8 glucose units in length. More specifically, β-glucan derived from baker's yeast is poly-(1,6)-8-D-glucopyranosyl-(1,3)-13-D-glucopyranose.

Furthermore, β-glucans are well tolerated and do not produce or cause excess gas, abdominal distension, bloating or diarrhea in pediatric subjects. Addition of β-glucan to a nutritional composition for a pediatric subject, such as an infant formula, a growing-up milk or another children's nutritional product, will improve the subject's immune response by increasing resistance against invading pathogens and therefore maintaining or improving overall health.

In some embodiments, the β-glucan is β-1,3;1,6-glucan. In some embodiments, the β-1,3;1,6-glucan is derived from baker's yeast. The nutritional composition may comprise whole glucan particle β-glucan, particulate δ-glucan, PGG-glucan (poly-1,6-β-D-glucopyranosyl-1,3-β-D-glucopyranose) or any mixture thereof.

In some embodiments, the amount of β-glucan in the nutritional composition is between about 3 mg and about 17 mg per 100 Kcal. In another embodiment the amount of β-glucan is between about 6 mg and about 17 mg per 100 Kcal.

The nutritional composition of the present disclosure may comprise lactoferrin in some embodiments. Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe3+) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pl 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms.

Lactoferrin for use in the present disclosure may be, for example, isolated from the milk of a non-human animal or produced by a genetically modified organism. The oral electrolyte solutions described herein can, in some embodiments comprise non-human lactoferrin, non-human lactoferrin produced by a genetically modified organism and/or human lactoferrin produced by a genetically modified organism.

Suitable non-human lactoferrins for use in the present disclosure include, but are not limited to, those having at least 48% homology with the amino acid sequence of human lactoferrin. For instance, bovine lactoferrin (bLF) has an amino acid composition which has about 70% sequence homology to that of human lactoferrin. In some embodiments, the non-human lactoferrin has at least 65% homology with human lactoferrin and in some embodiments, at least 75% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin.

In some embodiments, the nutritional composition of the present disclosure comprises non-human lactoferrin, for example bLF. bLF is a glycoprotein that belongs to the iron transporter or transferring family. It is isolated from bovine milk, wherein it is found as a component of whey. There are known differences between the amino acid sequence, glycosylation patters and iron-binding capacity in human lactoferrin and bLF. Additionally, there are multiple and sequential processing steps involved in the isolation of bLF from cow's milk that affect the physiochemical properties of the resulting bLF preparation. Human lactoferrin and bLF are also reported to have differences in their abilities to bind the lactoferrin receptor found in the human intestine.

Though not wishing to be bound by this or any other theory, it is believe that bLF that has been isolated from whole milk has less lipopolysaccharide (LPS) initially bound than does bLF that has been isolated from milk powder. Additionally, it is believed that bLF with a low somatic cell count has less initially-bound LPS. A bLF with less initially-bound LPS has more binding sites available on its surface. This is thought to aid bLF in binding to the appropriate location and disrupting the infection process.

bLF suitable for the present disclosure may be produced by any method known in the art. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

In certain embodiments, lactoferrin utilized in the present disclosure may be provided by an expanded bed absorption (EBA) process for isolating proteins from milk sources. EBA, also sometimes called stabilized fluid bed adsorption, is a process for isolating a milk protein, such as lactoferrin, from a milk source comprises establishing an expanded bed adsorption column comprising a particulate matrix, applying a milk source to the matrix, and eluting the lactoferrin from the matrix with an elution buffer comprising about 0.3 to about 2.0 M sodium chloride. Any mammalian milk source may be used in the present processes, although in particular embodiments, the milk source is a bovine milk source. The milk source comprises, in some embodiments, whole milk, reduced fat milk, skim milk, whey, casein, or mixtures thereof.

In particular embodiments, the target protein is lactoferrin, though other milk proteins, such as lactoperoxidases or lactalbumins, also may be isolated. In some embodiments, the process comprises the steps of establishing an expanded bed adsorption column comprising a particulate matrix, applying a milk source to the matrix, and eluting the lactoferrin from the matrix with about 0.3 to about 2.0M sodium chloride. In other embodiments, the lactoferrin is eluted with about 0.5 to about 1.0 M sodium chloride, while in further embodiments, the lactoferrin is eluted with about 0.7 to about 0.9 M sodium chloride.

The expanded bed adsorption column can be any known in the art, such as those described in U.S. Pat. Nos. 7,812,138, 6,620,326, and 6,977,046, the disclosures of which are hereby incorporated by reference herein. In some embodiments, a milk source is applied to the column in an expanded mode, and the elution is performed in either expanded or packed mode. In particular embodiments, the elution is performed in an expanded mode. For example, the expansion ratio in the expanded mode may be about 1 to about 3, or about 1.3 to about 1.7. EBA technology is further described in international published application nos. WO 92/00799, WO 02/18237, WO 97/17132, which are hereby incorporated by reference in their entireties.

The isoelectric point of lactoferrin is approximately 8.9. Prior EBA methods of isolating lactoferrin use 200 mM sodium hydroxide as an elution buffer. Thus, the pH of the system rises to over 12, and the structure and bioactivity of lactoferrin may be comprised, by irreversible structural changes. It has now been discovered that a sodium chloride solution can be used as an elution buffer in the isolation of lactoferrin from the EBA matrix. In certain embodiments, the sodium chloride has a concentration of about 0.3 M to about 2.0 M. In other embodiments, the lactoferrin elution buffer has a sodium chloride concentration of about 0.3 M to about 1.5 M, or about 0.5 m to about 1.0 M.

In other embodiments, lactoferrin for use in the composition of the present disclosure can be isolated through the use of radial chromatography or charged membranes, as would be familiar to the skilled artisan.

The lactoferrin that is used in certain embodiments may be any lactoferrin isolated from whole milk and/or having a low somatic cell count, wherein “low somatic cell count” refers to a somatic cell count less than 200,000 cells/mL. By way of example, suitable lactoferrin is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

Surprisingly, lactoferrin included herein maintains certain bactericidal activity even if exposed to a low pH (i.e., below about 7, and even as low as about 4.6 or lower) and/or high temperatures (i.e., above about 65° C., and as high as about 120° C.), conditions which would be expected to destroy or severely limit the stability or activity of human lactoferrin. These low pH and/or high temperature conditions can be expected during certain processing regimen for nutritional compositions of the types described herein, such as pasteurization. Therefore, even after processing regimens, lactoferrin has bactericidal activity against undesirable bacterial pathogens found in the human gut. The nutritional composition may, in some embodiments, comprise lactoferrin in an amount from about 25 mg/100 mL to about 150 mg/100 mL. In other embodiments lactoferrin is present in an amount from about 60 mg/100 mL to about 120 mg/100 mL. In still other embodiments lactoferrin is present in an amount from about 85 mg/100 mL to about 110 mg/100 mL.

The disclosed nutritional composition described herein, can, in some embodiments also comprise an effective amount of iron. The iron may comprise encapsulated iron forms, such as encapsulated ferrous fumarate or encapsulated ferrous sulfate or less reactive iron forms, such as ferric pyrophosphate or ferric orthophosphate.

One or more vitamins and/or minerals may also be added in to the nutritional composition in amounts sufficient to supply the daily nutritional requirements of a subject. It is to be understood by one of ordinary skill in the art that vitamin and mineral requirements will vary, for example, based on the age of the child. For instance, an infant may have different vitamin and mineral requirements than a child between the ages of one and thirteen years. Thus, the embodiments are not intended to limit the nutritional composition to a particular age group but, rather, to provide a range of acceptable vitamin and mineral components.

In embodiments providing a nutritional composition for a child, the composition may optionally include, but is not limited to, one or more of the following vitamins or derivations thereof: vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B₃ (niacin, nicotinic acid, nicotinamide, niacinamide, nicotinamide adenine dinucleotide, NAD, nicotinic acid mononucleotide, NicMN, pyridine-3-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D₃, 1,25,-dihydroxyvitamin D), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

In embodiments providing a children's nutritional product, such as a growing-up milk, the composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to growing-up milks or to other children's nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

The nutritional compositions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product. Indeed, the incorporation of dietary butyrate into a nutritional composition, such as an infant formula, may require the presence of at least on emulsifier to ensure that the dietary butyrate does not separate from the fat or proteins contained within the infant formula during shelf-storage or preparation.

In some embodiments, the nutritional composition may be formulated to include from about 0.5 wt % to about 1 wt % of emulsifier based on the total dry weight of the nutritional composition. In other embodiments, the nutritional composition may be formulated to include from about 0.7 wt % to about 1 wt % of emulsifier based on the total dry weight of the nutritional composition.

In some embodiments where the nutritional composition is a ready-to-use liquid composition, the nutritional composition may be formulated to include from about 200 mg/L to about 600 mg/L of emulsifier. Still, in certain embodiments, the nutritional composition may include from about 300 mg/L to about 500 mg/L of emulsifier. In other embodiments, the nutritional composition may include from about 400 mg/L to about 500 mg/L of emulsifier.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, potassium citrate, calcium disodium EDTA, and mixtures thereof. The incorporation of a preservative in the nutritional composition including dietary butyrate ensures that the nutritional composition has a suitable shelf-life such that, once reconstituted for administration, the nutritional composition delivers nutrients that are bioavailable and/or provide health and nutrition benefits for the target subject.

In some embodiments the nutritional composition may be formulated to include from about 0.1 wt % to about 1.0 wt % of a preservative based on the total dry weight of the composition. In other embodiments, the nutritional composition may be formulated to include from about 0.4 wt % to about 0.7 wt % of a preservative based on the total dry weight of the composition.

In some embodiments where the nutritional composition is a ready-to-use liquid composition, the nutritional composition may be formulated to include from about 0.5 g/L to about 5 g/L of preservative. Still, in certain embodiments, the nutritional composition may include from about 1 g/L to about 3 g/L of preservative.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof. Indeed, incorporating a suitable stabilizer in the nutritional composition including dietary butyrate ensures that the nutritional composition has a suitable shelf-life such that, once reconstituted for administration, the nutritional composition delivers nutrients that are bioavailable and/or provide health and nutrition benefits for the target subject.

In some embodiments where the nutritional composition is a ready-to-use liquid composition, the nutritional composition may be formulated to include from about 50 mg/L to about 150 mg/L of stabilizer. Still, in certain embodiments, the nutritional composition may include from about 80 mg/L to about 120 mg/L of stabilizer.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and contains suitable types and amounts of lipid, carbohydrate, protein, vitamins and minerals. The amount of lipid or fat typically can vary from about 1 to about 25 g/100 Kcal. The amount of protein typically can vary from about 1 to about 7 g/100 Kcal. The amount of carbohydrate typically can vary from about 6 to about 22 g/100 Kcal.

In an embodiment, the children's nutritional composition may contain between about 10 and about 50% of the maximum dietary recommendation for any given country, or between about 10 and about 50% of the average dietary recommendation for a group of countries, per serving of vitamins A, C, and E, zinc, iron, iodine, selenium, and choline. In another embodiment, the children's nutritional composition may supply about 10-30% of the maximum dietary recommendation for any given country, or about 10-30% of the average dietary recommendation for a group of countries, per serving of B-vitamins. In yet another embodiment, the levels of vitamin D, calcium, magnesium, phosphorus, and potassium in the children's nutritional product may correspond with the average levels found in milk. In other embodiments, other nutrients in the children's nutritional composition may be present at about 20% of the maximum dietary recommendation for any given country, or about 20% of the average dietary recommendation for a group of countries, per serving.

In some embodiments the nutritional composition is an infant formula. Infant formulas are fortified nutritional compositions for an infant. The content of an infant formula is dictated by federal regulations, which define macronutrient, vitamin, mineral, and other ingredient levels in an effort to simulate the nutritional and other properties of human breast milk. Infant formulas are designed to support overall health and development in a pediatric human subject, such as an infant or a child.

In some embodiments, the nutritional composition of the present disclosure is a growing-up milk. Growing-up milks are fortified milk-based beverages intended for children over 1 year of age (typically from 1-3 years of age, from 4-6 years of age or from 1-6 years of age). They are not medical foods and are not intended as a meal replacement or a supplement to address a particular nutritional deficiency. Instead, growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of a growing-up milk or other nutritional composition according to the present disclosure can vary from market-to-market, depending on local regulations and dietary intake information of the population of interest. In some embodiments, nutritional compositions according to the disclosure consist of a milk protein source, such as whole or skim milk, plus added sugar and sweeteners to achieve desired sensory properties, and added vitamins and minerals. The fat composition includes an enriched lipid fraction derived from milk. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, in some embodiments, vitamins and minerals can be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

The disclosed nutritional composition(s) may be provided in any form known in the art, such as a powder, a gel, a suspension, a paste, a solid, a liquid, a liquid concentrate, a reconstituteable powdered milk substitute or a ready-to-use product. The nutritional composition may, in certain embodiments, comprise a nutritional supplement, children's nutritional product, infant formula, human milk fortifier, growing-up milk or any other nutritional composition designed for an infant or a pediatric subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. In some embodiments, the nutritional composition is in powder form with a particle size in the range of 5 μm to 1500 μm, more preferably in the range of 10 μm to 300 μm.

The nutritional compositions of the present disclosure may be provided in a suitable container system. For example, non-limiting examples of suitable container systems include plastic containers, metal containers, foil pouches, plastic pouches, multi-layered pouches, and combinations thereof. In certain embodiments, the nutritional composition may be a powdered composition that is contained within a plastic container. In certain other embodiments, the nutritional composition may be contained within a plastic pouch located inside a plastic container.

In some embodiments, the method is directed to manufacturing a powdered nutritional composition. The term “powdered nutritional composition” as used herein, unless otherwise specified, refers to dry-blended powdered nutritional formulations comprising protein, and specifically plant protein, and at least one of fat and carbohydrate, which are reconstitutable with an aqueous liquid, and which are suitable for oral administration to a human.

Indeed, in some embodiments, the method comprises the steps of dry-blending selected nutritional powders of the nutrients selected to create a base nutritional powder to which additional selected ingredients, such as dietary butyrate, may be added and further blended with the base nutritional powder. The term “dry-blended” as used herein, unless otherwise specified, refers to the mixing of components or ingredients to form a base nutritional powder or, to the addition of a dry, powdered or granulated component or ingredient to a base powder to form a powdered nutritional formulation. In some embodiments, the base nutritional powder is a milk-based nutritional powder. In some embodiments, the base nutritional powder includes at least one fat, one protein, and one carbohydrate. The powdered nutritional formulations may have a caloric density tailored to the nutritional needs of the target subject.

The powdered nutritional compositions may be formulated with sufficient kinds and amounts of nutrients so as to provide a sole, primary, or supplemental source of nutrition, or to provide a specialized powdered nutritional formulation for use in individuals afflicted with specific diseases or conditions. For example, in some embodiments, the nutritional compositions disclosed herein may be suitable for administration to pediatric subjects and infants in order provide exemplary health benefits disclosed herein.

The powdered nutritional compositions provided herein may further comprise other optional ingredients that may modify the physical, chemical, hedonic or processing characteristics of the products or serve as nutritional components when used in the targeted population. Many such optional ingredients are known or otherwise suitable for use in other nutritional products and may also be used in the powdered nutritional compositions described herein, provided that such optional ingredients are safe and effective for oral administration and are compatible with the essential and other ingredients in the selected product form. Non-limiting examples of such optional ingredients include preservatives, antioxidants, emulsifying agents, buffers, additional nutrients as described herein, colorants, flavors, thickening agents and stabilizers, and so forth.

The powdered nutritional compositions of the present disclosure may be packaged and sealed in single or multi-use containers, and then stored under ambient conditions for up to about 36 months or longer, more typically from about 12 to about 24 months. For multi-use containers, these packages can be opened and then covered for repeated use by the ultimate user, provided that the covered package is then stored under ambient conditions (e.g., avoid extreme temperatures) and the contents used within about one month or so.

In some embodiments, the method further comprises the step of placing the nutritional compositions in a suitable package. A suitable package may comprise a container, tub, pouch, sachet, bottle, or any other container known and used in the art for containing nutritional composition. In some embodiments, the package containing the nutritional composition is a plastic container. In some embodiments, the package containing the nutritional composition is a metal, glass, coated or laminated cardboard or paper container. Generally, these types of packaging materials are suitable for use with certain sterilization methods utilized during the manufacturing of nutritional compositions formulated for oral administration.

In some embodiments, the nutritional compositions are packaged in a container. The container for use herein may include any container suitable for use with powdered and/or liquid nutritional products that is also capable of withstanding aseptic processing conditions (e.g., sterilization) as described herein and known to those of ordinary skill in the art. A suitable container may be a single-dose container, or may be a multi-dose resealable, or reclosable container that may or may not have a sealing member, such as a thin foil sealing member located below the cap. Non-limiting examples of such containers include bags, plastic bottles or containers, pouches, metal cans, glass bottles, juice box-type containers, foil pouches, plastic bags sold in boxes, or any other container meeting the above-described criteria. In some embodiments, the container is a resealable multi-dose plastic container. In certain embodiments, the resealable multi-dose plastic container further comprises a foil seal and a plastic resealable cap. In some embodiments, the container may include a direct seal screw cap. In other embodiments, the container may be a flexible pouch.

In some embodiments, the nutritional composition is a liquid nutritional composition and is processed via a “retort packaging” or “retort sterilizing” process. The terms “retort packaging” and “retort sterilizing” are used interchangeably herein, and unless otherwise specified, refer to the common practice of filling a container, most typically a metal can or other similar package, with a nutritional liquid and then subjecting the liquid-filled package to the necessary heat sterilization step, to form a sterilized, retort packaged, nutritional liquid product.

In some embodiments, the nutritional compositions disclosed herein are processed via an acceptable aseptic packaging method. The term “aseptic packaging” as used herein, unless otherwise specified, refers to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product.

The nutritional compositions described herein, in some embodiments, advantageously promote healthy weight maintenance in target subject and reduce the incidence of obesity and metabolic syndrome in target subjects. Indeed, early nutrition is important for healthy metabolic programming. Healthy weight and metabolism require a tight interplay between organs. Without being bound by any particular theory, dietary butyrate is thought to assist with early cellular and tissue programming that provides healthy body composition and metabolism and of associated tissues, such as brain tissue. Further, provided are methods for improving adipose tissue functioning and/or improving the quality of adipose tissue in target subjects. Further provided are methods for reducing the incidence of obesity and metabolic syndrome in target subjects. Also provided are methods for supporting, reinforcing, or improving healthy weight and metabolism in target subjects who do not have a healthy weight or metabolism. In some embodiments, the target subject is a formula-fed infant or a pediatric subject that was formula-fed.

In some embodiments, the method comprises the step of administering the nutritional composition disclosed herein comprising dietary butyrate to the target subject. Indeed, in certain embodiments where the target subject is a formula-fed infant, the formula-fed infant will experience an improvement in adipose tissue function and quality as compared to other formula-fed infants that are not provided the nutritional composition including dietary butyrate.

In some embodiments the target subject may be a pediatric subject. Further, in one embodiment, the nutritional composition provided to the pediatric subject may be an infant formula. In certain embodiment, the dietary butyrate may be formulated in an infant formula together with other ingredients, such as DHA, ARA, lactoferrin, PE, sphingomyelin, inositol, ALA, EGCG, sulforaphane, vitamin D, and combinations thereof. Without being bound by any particular theory the combination of dietary butyrate together with these selected ingredients may act synergistically and provide synergistic health benefits to the target subject.

Further provided are methods of a staged infant feeding regimen to promote healthy development and growth, such as increased and/or accelerated myelination, in a formula-fed infant. In certain embodiments, the feeding regimen includes feeding a first nutritional composition, a second nutritional composition, and a third nutritional composition to an infant as the infant ages. In the respective staged feeding regimen disclosed herein, each of the first, second, third, etc. nutritional compositions are different from each other.

Indeed, while a conventional “one size fits all” infant formula can provide adequate nutrition for a formula-fed infant, such formulas do not account for the changing requirements during development. Accordingly, it would be beneficial to provide an infant feeding regimen which includes nutritional compositions tailored to provide a combination of nutrients designed to promote healthy development and growth at each stage. Furthermore, it is beneficial to provide staged nutritional compositions tailored to continue promoting and accelerating myelination in a formula-fed infant throughout the first 12 months of life.

“Infant” is a human from birth through not more than 12 months of age, where a “newborn infant” is an infant from birth through 3 months of age, a “middle stage infant” is an infant from 3 months of age through 6 months of age, and a “later stage infant” is an infant from 6 months of age through 12 months of age or 1 year.

Accordingly, in certain embodiments, the first nutritional composition is fed to an infant that is a newborn infant, i.e. age birth through 3 months of age, the second nutritional composition is fed to a middle stage infant, i.e. an infant that is age 3 months of age to 6 months of age, and the third composition is fed to a later stage infant, i.e. an infant that is age 6 months of age to 12 months of age.

In certain embodiments, the feeding regimen of the present disclosure includes:

A) a first nutritional composition comprising:

i) up to about 0.2 mg/100 kcal of iron;

ii) up to about 0.7 mg/100 kcal of zinc;

iii) up to about 2 g/100 kcal of protein;

iv) a whey:casein ratio of at least 72:28 and, in embodiments, between 72:28 and 90:10;

v) up to 35 mg/100 kcal of inositol;

vi) up to 9.0 mg/100 kcal of butyrate; and/or

vii) from about 1.8 μg/100 kcal to about 2.0 μg/100 kcal of vitamin D;

B) a second nutritional composition comprising:

i) from about 0.3 mg/100 kcal to about 0.6 mg/100 kcal of iron;

ii) up to about 0.9 mg/100 kcal of zinc;

iii) up to about 1.9 g/100 kcal of protein;

iv) a whey:casein ratio of between 56:44 and 68:32;

v) up to 25 mg/100 kcal of inositol;

vi) up to 9.0 mg/100 kcal of butyrate; and/or

vii) from about 1.8 μg/100 kcal to about 2.0 μg/100 kcal of vitamin D; and

C) a third nutritional composition comprising:

i) from about 0.3 mg/100 kcal to about 1.2 mg/100 kcal of iron;

ii) up to about 1 mg/100 kcal of zinc;

iii) up to about 1.5 g/100 kcal of protein;

iv) a whey:casein ratio of between 45:55 and 68:32;

v) up to 20 mg/100 kcal of inositol;

vi) up to 9.0 mg/100 kcal of butyrate; and/or

vii) from about 1.8 μg/100 kcal to about 2.0 μg/100 kcal of vitamin D.

In certain embodiments, the feeding regimen of the present disclosure includes:

A) a first nutritional composition comprising:

i) up to about 0.45 mg/100 kcal of iron;

ii) up to about 0.7 mg/100 kcal of zinc;

iii) up to about 2 g/100 kcal of protein;

iv) a whey:casein ratio of at least 72:28 and, in embodiments, between 72:28 and 90:10;

v) up to 35 mg/100 kcal of inositol;

vi) up to 9.0 mg/100 kcal of butyrate; and/or

vii) from about 1.8 μg/100 kcal to about 2.0 μg/100 kcal of vitamin D;

B) a second nutritional composition comprising:

i) from about 0.3 mg/100 kcal to about 0.6 mg/100 kcal of iron;

ii) up to about 0.9 mg/100 kcal of zinc;

iii) up to about 1.9 g/100 kcal of protein;

iv) a whey:casein ratio of between 56:44 and 68:32;

v) up to 25 mg/100 kcal of inositol;

vi) up to 9.0 mg/100 kcal of butyrate; and/or

vii) from about 1.8 μg/100 kcal to about 2.0 μg/100 kcal of vitamin D; and

C) a third nutritional composition comprising:

i) from about 0.6 mg/100 kcal to about 1.2 mg/100 kcal of iron;

ii) up to about 2 mg/100 kcal of zinc;

iii) up to about 3 g/100 kcal of protein;

iv) a whey:casein ratio of between 45:55 and 54:46;

v) up to 20 mg/100 kcal of inositol;

vi) up to 9.0 mg/100 kcal of butyrate; and/or

vii) from about 1.8 μg/100 kcal to about 2.0 μg/100 kcal of vitamin D.

In certain embodiments, the feeding regimen of the present disclosure includes:

A) a first nutritional composition comprising:
i) no greater than 9.0 mg/100 kcal of butyrate; and

B) a second nutritional composition comprising:
i) at least 9.0 mg/100 kcal of butyrate.
   Indeed, in this example embodiment the level of butyrate is increased from the first composition compared to the second composition. Indeed, further, the first composition may be provided to an infant from the age of birth to three months and the second composition is then fed to the infant when the infant is age 3 months to 12 months. Indeed, the level of butyrate is increased as the infant increases in age. Further, in some embodiments the first composition comprises other nutrients such as sialic acid, human milk oligosaccharides, and/or lactoferrin. Indeed, In some embodiments the levels of these additional nutrients are actually higher in the first composition as compared to the second composition. Accordingly, while the concentration of dietary supplied butyrate is increased as the infant ages, the concentration of these other nutrients is decreased as the infant ages.

In certain embodiments, the feeding regimen of the present disclosure includes: A) a first nutritional composition comprising from about 7 μg/mL to about 22 μg/mL of butyrate; B) a second nutritional composition comprising from about 17 μg/mL to about 24 μg/mL of butyrate; and C) a third nutritional composition comprising from about 17 μg/mL to about 22 μg/mL of butyrate.

In certain embodiments, the feeding regimen of the present disclosure includes: A) a first nutritional composition comprising from about 5 μg/mL to about 30 μg/mL of butyrate; B) a second nutritional composition comprising from about 15 μg/mL to about 25 μg/mL of butyrate; and C) a third nutritional composition comprising from about 17 μg/mL to about 22 μg/mL of butyrate.

Indeed, in some embodiments the first nutritional composition is administered to an infant from birth to the age of 6 months, the second nutritional composition is administered to an infant from 6 months to the age of 12 months, and the third nutritional composition is administered to an infant/child after the age of 12 months.

Also provided is a staged feeding regimen for an infant or child where the infant is administered a series of nutritional compositions comprising

A) a first nutritional composition comprising from about 7 μg/mL to about 10 μg/mL of dietary butyrate that is administered to the infant from birth to the age of two weeks;

B) a second nutritional composition comprising from about 9 μg/mL to about 13 μg/mL of dietary butyrate that is administered to the infant from the age of two weeks to 4 weeks;

C) a third nutritional composition comprising from about 16 μg/mL to about 22 μg/mL of dietary butyrate that is administered to the infant from the age of 4 weeks to 13 weeks;

D) a fourth nutritional composition comprising from about 17 μg/mL to about 24 μg/mL of dietary butyrate that is administered to the infant from the age of 13 weeks to about 26 weeks; and

E) a fifth nutritional composition comprising from about 17 μg/mL to about 22 μg/mL of dietary butyrate that is administered to the infant from the age of 26 weeks to about 52 weeks.

Indeed, in certain embodiments, the feeding regimen is designed to provide the highest concentration of dietary butyrate to the infant when the infant is at least 26 weeks old.

In certain embodiments, the first nutritional composition may have a whey:casein ratio of about 80:20; in other embodiments, the first nutritional composition may have a whey:casein ratio of from about 72:28 to about 90:10.

In certain embodiments, the second nutritional composition may have a whey:casein ratio of from about 60:40. In other embodiments, the second nutritional composition may have a whey:casein ratio of from about 56:44 to about 68:32. Still, in certain embodiments, the second nutritional composition may have a whey:casein ratio of from about 56:44 to about 70:30.

In certain embodiments, the third nutritional composition may have a whey:casein ratio of from about 50:50. In other embodiments, the third nutritional composition may have a whey:casein ratio of from about 45:55 to about 54:46.

### Example 2

This example illustrates an embodiment of the feeding regimen of the present disclosure. Each nutritional composition disclosed herein may include other nutrients provided herein that are not listed in the Tables below. Provided below is an example of a first nutritional composition.

| Description | UOM | per 100 kcal |
|---|---|---|
| Iron | mg | 0.2 |
| Zinc | mg | 0.7 |
| Protein | g | 2.0 |
| Whey:Casein Ratio | | 80:20 |
| Inositol | mg | 35 |
| Butyrate | mg | 9.0 |
| Vitamin D | μg | 1.8-2.0 |

Provided below is an example of a second nutritional composition.

| Description | UOM | per 100 kcal |
|---|---|---|
| Iron | mg | 0.3-0.6 |
| Zinc | mg | 0.9 |
| Protein | g | 1.9 |
| Whey:Casein Ratio | | 60:40 |
| Inositol | mg | 25 |
| Butyrate | mg | 9 |
| Vitamin D | μg | 1.8-2.0 |

Provided below is an example of a third nutritional composition.

| Description | UOM | per 100 kcal |
|---|---|---|
| Iron | mg | 0.3-1.2 |
| Zinc | mg | 3.0 |
| Protein | g | 1.5 |
| Whey:Casein Ratio | | 50:50 |
| Inositol | mg | 20 |
| Butyrate | mg | 9.0 |
| Vitamin D | μg | 1.8-2.0 |

### Example 3

This example illustrates an embodiment of the feeding regimen of the present disclosure. Each nutritional composition disclosed herein may include other nutrients provided herein that are not listed in the Tables below. Provided below is an example of a first nutritional composition.

| Description | UOM | per 100 kcal |
|---|---|---|
| Iron | mg | 0.46 |
| Zinc | mg | 0.7 |
| Protein | g | 2.0 |
| Whey:Casein Ratio | | 80:20 |
| Inositol | mg | 35 |
| Butyrate | mg | 9 |
| Vitamin D | μg | 1.8-2.0 |

Provided below is an example of a second nutritional composition.

| Description | UOM | per 100 kcal |
|---|---|---|
| Iron | mg | 0.3-0.6 |
| Zinc | mg | 0.9 |
| Protein | g | 1.9 |
| Whey:Casein Ratio | | 60:40 |
| Inositol | mg | 25 |
| Butyrate | mg | 9 |
| Vitamin D | μg | 1.8-2.0 |

Provided below is an example of a third nutritional composition.

| Description | UOM | per 100 kcal |
|---|---|---|
| Iron | mg | 0.6-1.2 |
| Zinc | mg | 2 |
| Protein | g | 3 |
| Whey:Casein Ratio | | 50:50 |
| Inositol | mg | 20 |
| Butyrate | mg | 9 |
| Vitamin D | μg | 1.8-2.0 |

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

### Example 4

Example 4 illustrates a method for accurately determining the level of butyrate present in samples of human breast milk. Indeed, limited references are available discussing the levels of butyrate found in human breast milk and often reported concentration levels of butyrate in human breast milk differs significantly from reports in literature. Also, butyrate can be present in its free form as butyric acid or bound with a triacylglycerol molecule. As such, it is also unclear from the current literature if total butyrate is reported or if only the free form of butyrate is reported.

The following Definitions may be used in Example 4.

TABLE 5
Definitions

| Term | Definitions |
|---|---|
| BA | Butyric Acid |
| BA-D7 | Butyric Acid (D7) deuterated |
| IS | Internal standard |
| NPH₂O | Nanopure water |
| N/A | Not available |
| RT | Room temperature |
| CoA | Certificate of Analysis |
| EtAc | Ethyl Acetate |
| H₃PO₄ | Phosphoric acid |
| CRL | Lipase from Candida rugose |
| PBS | 0.1 Phosphate Buffered Saline |
| SSA | 5-sulfosalicylic acid |
| HM | Human milk |
| GC | Gas chromatography |
| MSD | Mass Spectrometer Detector |
| EI | Electron ionization |
| GC-MS | Gas Chromatography combined |
| | with Mass Spectrometer |
| Min | Minute |
| O/N | Overnight |
| Ex | Example |

The following Equipment and Apparatus may be used in Example 4. Note, equivalents may be used unless otherwise specified.

TABLE 6
Equipment & Apparatus

| Equipment | Description (brand, make, model, etc) |
|---|---|
| Gas Chromatography | Agilent 7890B series GC |
| Mass spectrometer | Agilent 5977A inert EI Source MSD |
| Hydrogen generator | Parker Hannifan Corp. H2PD-300 |
| Autosampler | Agilent 7693A autoinjector and tray |
| Analytical column | Phenomenex Zebron ZB-FFAP, 30 m × 0.5 mm × 1.00 μm |
| Balance | Mettler Toledo with anti-static kit or equivalent. Sensibility of |
| | balance to ±0.1 mg |
| Centrifuge | Sorvall Biofuge Stratos-Model#LR56495, Relabive force to 10,000 G |
| Vortex mixer | VWR Analog vortex mixer (Model #VM-3000) or equivalent |
| Thermo mixer C | Eppendorf Thermo Mixer C with Thermo Top |
| Sonicator | Fisher SolidState/Ultrasonic FS-28 |
| Pipettor | Biohit |
| Pipette tips | Biohit Optifit 10 μL-1 mL or equivalent |
| 1.5 mL screw cap tubes | Sarstedt #72692 1.5 mL polypropylene with screw cap, |
| | centrifugation to 20,000 × g |
| 1.5 mL tubes | Eppendorf Protein Lo-bind 1.5 mL tubes #022431081 |
| Autosampler vials | Agilent 12 × 32 mm, clear screw top GC 2 mL vials #5183-2067 |
| Silicone septa screw caps | Agilent PTFE; silicone/PTFE septa screw cap #5185-5862 |
| Glass insert | Agilent 250 μL deactivated glass insert with polymer feet #5181-8872 |
| Stir plate | Corning PC-351 Hotplate-Stirrer |
| 10 μL, 25 μL, 50 μL, 100 μL | Gas tight syringes |

The following Chemicals and Reagents may be used in Example 4.

TABLE 7
Chemicals and Reagents

| No. | Reagent Name | Concentration | Preparation No. | Brand | Shelf life | Storage |
|---|---|---|---|---|---|---|
| 1 | NP water | 18.2 MΩ/cm | N/A | Barnstead | 1 month | RT |
| 2 | BupH PBS | 0.1 M | 1 | Thermo #28372 | 2 weeks | RT |
| 3 | IS BA-D7 | Neat | N/A | Cambridge isotope | 1 year | 4° C. |
| | | | | DLM-1508-5 | | |
| 4 | IS BA-D7 | 1 mg/mL in NPH₂O | 2 | N/A | Daily | RT |
| 5 | IS BA-D7 | 1 mg/mL in EtAc | V.A-2 | N/a | Monthly | −20° C. |
| 6 | CRL | Powder | N/A | Sigma I8525-5 mu | 1 year | N/A |
| 7 | CRL | 3 mg/mL | 3 | N/A | Daily | N/A |
| 8 | BA | Neat | N/A | Sigma 19215 | 1 year | 2 |
| 9 | BA | V.A-1 | V.A-1 | N/A | Monthly | 2 |
| 10 | SSA | Powder | N/A | Sigma S7422 | 3 years | 3 |
| 11 | SSA | 1 mg/μL | 4 | N/A | Make daily | N/A |
| 12 | o-phosphoric acid | ≥85% | N/A | Fisher Scientific, | 3 years | 4 |
| | (ACS grade) | | | A242-500 | | |
| 13 | EtAc | 99.9% | N/A | Sigma #650528 | 1 year | 5 |
| 14 | Innovative | N/A | N/A | Innovative Research | 10 years | 6 |
| | human milk | | | #IR1000042 | | |
| 15 | Control- | N/A (aliquot) | 5.6 | Innovative Research | 10 years | 6 |
| | Innovative | | | #IR1000042 | | |
| | human milk | | | | | |

Accordingly, based on multiple questions regarding total butyrate levels in human milk and gaps found in the literature, Example 4 was developed and utilizes a nonspecific microbial triacylglycerol lipase form the yeast family to hydrolyze bound butyrate to free butyric acid, which is then deproteinized, acidified, and extracted into an organic solvent. The butyric acid in the extract is separated and identified from other compounds on a polar capillary column and analyzed by Gas Chromatography combined with Mass Spectrometry (GC-MS). Due to the volatility of butyric acid, and internal standard of deuterated butyric acid (BA-D7) is used during quantification.

The human milk samples are prepared according the following procedure. A 500 mL solution of 0.1M PBS was prepared according to the following steps. Add 1 pouch of BupH PBS to a 500 mL bottle, QS to 500 mL with NPH₂O, add a stir bar, and mix on a stir plate until the pouch contents are dissolved. The resulting solution is a 0.1 M PBS solution having a pH of 7.2 when the pouch contents are dissolved in the final volume of 500 mL NPH₂O.

An internal standard of BA-D7 was prepared according to the following steps. Tare a 2 mL GC vial with screw cap, remove the cap and add 1.5 μL IS BA-D7 by pipette. Replace the cap and accurately weigh the IS, record weight. Multiply weight by purity, subtract the volume and re-suspend with NPH₂O at 1 mg/mL.

A lipase from Candida rugosa (CRL) of 3 mg/mL in PBS was prepared according to the following steps. First, the total amount of lipase from Candida rugosa was calculated based on the total number of samples and controls being prepared. For example, (10 samples+1 control)×240 μg lipase=2.64 mg of lipase. Also, 240 μg of lipase was accounted for for each sample in order to account for any pipetting loss. Once the amount of lipase needed is determined, resuspend the lipase in PBS at 3 mg/mL. Next, weigh out the total amount of lipase needed in a 1.5 mL protein Lo-bind tubes, record the weight of the CRL. Store on ice until needed, Add calculated volume of 0.1 MPBS, vortex, heat on Thermo mixer at 37 deg. C. at 900 RPM for 30 minutes to get into solution.

A 5-sulfosalicylic acid (SAA) 1 mg/μL in water solution was prepared according to the following steps. Calculated the total amount of SSA needed for the number of samples and control being prepared. For example, (10 samples+1 control)×14 mg SSA=154 mg of SSA. Weigh out the total amount needed in a 1.5 mL protein Lo-bind tube, record the SSA weight. Re-suspend the SSA with NPH₂O at 1 mg/μL, for example 154 mg of SSA would be re-suspended with 154 μL of NPH₂O.

Human milk samples were prepared according to the following steps. Thaw a 50 mL container of Innovative Human milk over night at 4 deg. C. with gentle rocking, vortex, and aliquot 0.750 mL into pre-chilled 1.5 mL screw cap tubes. Vortex 50 mL container every sixth aliquot to make sure milk control sample stays mixed, then store in 1.5 mL tubes in −80 deg. C. Pull 2 mL of human milk samples and control from −80 deg. C. thaw in 4 deg. C. for about one hour, then put at room temperature until completely thawed. Invert 10 times to mix and vortex.

The butyric acid standard was prepared according to the following procedure. Make a 1 mg/mL solution of butyric acid standard in ethyl acetate. Tare a 5 mL volumetric flask with glass stopper, add 5.2 μL butyric acid with a pipette, weight flask and butyric acid, record butyric acid weight, QS flask to 5 mL, invert 20 times to mix. Calculate mg/mL using purity from Certificate of Analysis. For example, if the actual weight of butyric acid is 5.04 mg, then actual weight 5.04 mg×99.06% purity=5.02 mg/5 mL 1 mg/mL=1 μg/μL. Store in −20 deg. C. The butyric acid standards of 1 mg/μL need to be prepared monthly.

An internal standard BA-D7 procedure is prepared according to the following. Make a 1 mg/mL solution of the internal standard BA-D7 in ethyl acetate. Tare a 5 mL volumetric flask with a gas stopper, add 4.9 μL BA-D7 with a pipette, weigh flask and BA-D7, record BA-D7 weight. QS flask to 5 mL, invert 20 times to mix. Calculate mg/mL using purity from the Certificate of Analysis. For Example: actual weight 5.07 mg×98.5% purity=4.99 mg/5 mL, 1.00 mg/mL=1 μg/μL. Store in −20 deg. C. The internal standard BA-D7 1 mg/mL needs to be prepared monthly.

TABLE 8
Preparation of working butyric acid + internal
standard BA-D7 calibration standard curve

| Calibration | | | | | | | | | | |
|---|---|---|---|---|---|---|---|---|---|---|
| points | 1 | | 2 | | 3 | | 4 | | 5 | |
| Butyric acid | 1 | μg | 10 | μg | 50 | μg | 100 | μg | 150 | μg |
| amount | | | | | | | | | | |
| needed | | | | | | | | | | |
| IS BA-D7 | 15 | μg | 15 | μg | 15 | μg | 15 | μg | 15 | μg |
| amount | | | | | | | | | | |
| needed | | | | | | | | | | |
| Ethyl | QS 1 | mL | QS 1 | mL | QS 1 | mL | QS 1 | mL | QS 1 | mL |
| Acetate | | | | | | | | | | |

TABLE 9
Example preparation of working BA + IS BA-D7 calibration standard curve

| Calibration points | 1 | | 2 | | 3 | | 4 | | 5 | |
|---|---|---|---|---|---|---|---|---|---|---|
| BA volume | 1 | μL | 10 | μL | 50 | μL | 100 | μL | 150 | μL |
| from 1 mg/mL | | | | | | | | | | |
| IS BA-D7 | 15 | μL | 15 | μL | 15 | μL | 15 | μL | 15 | μL |
| volume from | | | | | | | | | | |
| 1 mg/mL | | | | | | | | | | |
| Ethyl | 984 | μL | 975 | μL | 935 | μL | 885 | μL | 835 | μL |
| acetate 1 mL | | | | | | | | | | |

Prepare the above 5 point working calibration standard curve in GC screw top vials, use gas tight syringes to add BA 1 mg/mL, IS BA-D7 1 mg/mL and ethyl acetate. Vortex to mix and store in −20 deg. C. until needed. Note: working calibration standards need to be prepared weekly.

Next, pull working calibration standards 1-5 from −20 deg. C., allow standards to warm to room temperature (approximately 20-30 minutes), vortex to mix. Transfer 150 μL to labeled GC vial with glass insert (note working standard 3 requires 3 replicates, (1) calibration standard curve, (1) after sixth sample to check for drift, and (1) at the end of the analyitcal run to check for drift).

### Sample Preparation—Human Milk

For each sample and control label a 1.5 mL screw cap tubes, up to 11 samples and 1 control. Pipette 500 μL human milk (HM) samples and control into the labeled 1.5 mL screw cap tube. Add 7.5 μL 1 mg/mL IS BA-D7 with gas tight syringe, vortex 10 sec. The internal standard is being added at 15 μg/mL, since the sample volume of HM is 0.5 mL, 7.5 μg is added. Sonicate the samples, control and CRL for 5 minutes at room temperature. Add 240 μg of lipase. Pipette 80 μL 3 mg/mL CRL solution into each sample and control, vortex 10 sec., and place on Thermo mixer at 37 deg. C. 900 RPM for at least 17 hours. Note: if the samples are put on at 4 pm, they come off the Thermo mixer at 9:00 am. Pull samples and control from the Thermo mixer, vortex and place on ice for 5 minutes. Add 14 μL 1 mg/μL SSA to each sample and control, vortex 10 seconds and incubate at room temperature for 10 minutes. Under a hood, add 30 μL H₃PO₄ to each sample and control, vortex 10 sec, then put on vortex mixer 5 minutes at 1300 RPM. Note: do not invert the 1.5 mL tubes after removing tubes from vortex in order to prevent sample loss in the cap during the next step. Under hood, add 500 μL ethyl acetate to each sample and control, vortex for 10 sec. then put on vortex mixer for 5 minutes at 1300 RPM. Centrifuge at 14,000 RPM, which is equal to 18,188×g, for 45 minutes at room temperature. Transfer 200 μL of top organic layer to labeled screw top GC vial with glass insert. Store remaining sample in −20 deg. C. Put GC vials in appropriate position on auto sampler.

### Analysis of Samples

TABLE 10
Set up for Sample Analysis

| Step | Task |
|---|---|
| 1 | Instrument Set-up: The GC-MS system should be located in an area |
| | where temperature fluctuations will be minimal throughout the run. |
| 2 | Load the test method SCFA_MJN_MS.M and let the instruments |
| | equilibrate for at least 20 minutes before running samples. |
| 3 | Set up GC-MS instrument parameters according to the next table. |

TABLE 11
Set-up for GC-MS

| Parameter Name | Instrument Set-up values | | | | |
|---|---|---|---|---|---|
| Column | Phenomenex Zebron ZB-FFAP, | | | | |
| | 30 m × 0.32 mm × 100 μm | | | | |
| Carry gas flow rate | Hydrogen at 2.8 mL/min. constant flow | | | | |
| Injector temp. | 250° C. (200° C. for standby mode) | | | | |
| Injection volume | 1 μL | | | | |
| Injection mode | Splitless | | | | |
| Transfer line | 250° C. (200° C. for standby mode) | | | | |
| temperature | | | | | |
| Mass | Source at 230° and MS Quad at 150° | | | | |
| spectrometer | | | | | |
| Temperature | | Rate | Value | Hold | Run |
| Program | Initial | (° C./min) | (° C.) | time min | time Min |

SCFA_MJN_MS.M

| | | | | |
|---|---|---|---|---|
| Initial | | 50 | 0 | 0 |
| Ramp 1 | 20 | 120 | 0 | 3.5 |
| Ramp 2 | 3 | 125 | 0 | 5.17 |
| Ramp 3 | 1 | 130 | 0 | 10.17 |
| Ramp 4 | 15 | 250 | 19 | 37.17 |

SCFA_Blank_MS.M

| | | | | |
|---|---|---|---|---|
| Initial | | 70 | 0 | 0 |
| Ramp 1 | 25 | 250 | 18 | 25.20 |

Standby_MS1column.M

| | | | |
|---|---|---|---|
| Initial | 70 | 10 | 10.0 |

TABLE 12
Sample Sequence Set up

| Step | Task |
|---|---|
| 1 | Sample Sequence Set up: |
| | Once the GC-MS instrument is equilibrated, inject a ″blank″ sample of ethyl |
| | acetate solution using SCFA_BLANK_MS.M method followed by another blank |
| | of ethyl acetate solution using method SCFA_MJN_MS.M. |
| 2 | Then inject each BA standard level (one time) one after another and |
| | begin from low to high concentration using method SCFA_MJN_MS.M. |
| 3 | Follow the last BA standard injection with one blank of ethyl acetate |
| | solution injection using method SCFA_Blank_MS.M. |
| 4 | Then inject 6 samples (one time) sequentially using method SCFA_MJN_MS.M. |
| 5 | Follow each sample inject one blank of ethyl acetate solution using |
| | method SCFA_Blank_MS.M. |
| 6 | Follow the blank injection atfter the 6^{th} sample or less run a middle level of BA |
| | standard using method SCFA_MJN_MS.M. |
| 7 | Then inject a blank of ethyl acetate solution at the end of the sequence. |
| 8 | If more than 6 samples are to be run, a middle level of BA standard have to be |
| | run again at the end of the sequence and then followed by a solvent blank. |

TABLE 13
End of Run Procedure

| Step | Task |
|---|---|
| 1 | End of Run: |
| | After all samples and standards have been analyzed, check the |
| | last blank run. If needed, following the MS baking procedure to |
| | bake the ionization source before loading the instrument standby |
| | method. |
| 2 | The capillary column in the GC should always be flown with |
| | carrier gas. |
| 3 | Turn the instrument from operation to standy mode when the |
| | instrument is not in use by loading the method |
| | Standby_MSl_column.M |
| 4 | If the instrument will be used in the next 2 hours, then |
| | keep the instrument in operational mode with method |
| | SCFA_MJN_MS.M loaded. |

TABLE 14
System Suitability

| Step | Parameter |
|---|---|
| 1 | SYSTEM SUITABILITY: |
| | Correlation coefficient R of standard curve |
| | should be a minimum of 0.99. |
| 2 | Retention times must be within ±10% of |
| | agreement between the standard and the samples. |
| 3 | Run an innovative HM as check every time |
| | samples are run. |

### Calculation and Report

Follow the instrument calibration process to get butyric acid result directly or use the following calculations to determine the butyric acid (BA) concentration:

a. Use Microsoft Excel of qualified chromatography software to plot a linear internal calibration curve using the ratio of standard concentration to internal standards versus the ratio of standard integration area to internal standards in the final extracts. Display the regression trendline equation (y=a*(C_{BA}/C_{IS})+b) and R-squared value to the curve. The R-squared value should be ≥0.990.

b. Calculate the butyric acid concentration (μg/mL) in the sample solution using linear regression trendline equation displayed in the Excel calculation sheet from step Via, the sample and extraction solvent volumes considered. Solve for the C_{BA}:

$${C}_{BA} = {( y - b )}^{*} {C}_{IS} * {V}_{EtAc} / ( {a}^{*} {V}_{sample} )$$

C_{BA}=Concentration of BA in the sample (μg/mL)

y=Ratio of BA peak area to internal standards (IS) in the injected sample solution

b=y-intercept of the curve

C_{IS}=concentration of IS spiked into the sample (μg/mL)

V_{EtAc}=Volume (μL) of solvent ethyl acetate (EtAc) used for extraction

a=slope of the curve

Vₛₐₘₚₗₑ=Sample volume (μL)

c. The BA final results should be reported in (μg/mL). Use equation above to calculate results or get the results using instrument software calibrations.

The blank and 5 point calibration standards can be loaded on the autosampler and the analytical run started before beginning sample extraction. In the sample sequence set up, there is a blank between each sample to prevent carryover, and a standard and blank after the sixth (6^{th}) and last sample to check for drift. All tips and 1.5 mL tubes used to analyze human milk are considered a biological hazard and are autoclaved before disposal. Hydrogen (H₂) is flammable, be aware that H₂ gas can flow into the GC oven and create an explosion hazard. Always turn off the H₂ supply before working on the instrument, ensure that the inlet and detector column fittings are connected to a column or capped at all times when H₂ gas is supplied to the instrument.

IS BA-D7 is a combustible liquid, contact can cause severe burns. Work in the fume hood or in a well-ventilated area and wear safety gloves, lab coat and protective eye glasses. Buytric acid (BA) is a combustible liquid, skin or eye contact can cause severe burns. Work in the fume hood or in a well-ventilated area and wear safety gloves, lab coat and protective eye glasses. 5-Sulfosalicylic acid (SSA) is a white powder to crystals, skin or eye contact can cause severe burns. Wear safety gloves, lab coat and protective eye glasses. Phosphoric acid 85% is corrosive and has a strong pungent odor, and contact can cause severe burns. Work in the fume hood or in a well-ventilated area and wear safety gloves, lab coat and protective eye glasses. Ethyl Acetate is highly flammable, it is harmful if inhaled or in contact with skin. Wear protective clothing, gloves and eye glasses. Work in the fume hood or in a well-ventilated area. Keep ethyl acetate away from heat and open flame. When working with human milk (HM) wear safety gloves, lab coat and protective eye glasses. Autoclave or bleach all tips, tubes, and lab supplies that come into contact with HM.

Method precision and accuracy: Precision: 1.8%-4.9% RSD; Accuracy: 83.7%-90.2%. Limit of detection and working range: LOD: 0.7 μg/mL; Range: 1 to 150 μg/mL. General References used: Method validation protocol 2016-001-MVP-AS.2002 vl and Report 2016-MVR-AS.2002 vl for the method AS.2002 vl: General Method for Determination of Butyrate in Human Milk using Internal Standard by GC-MS.

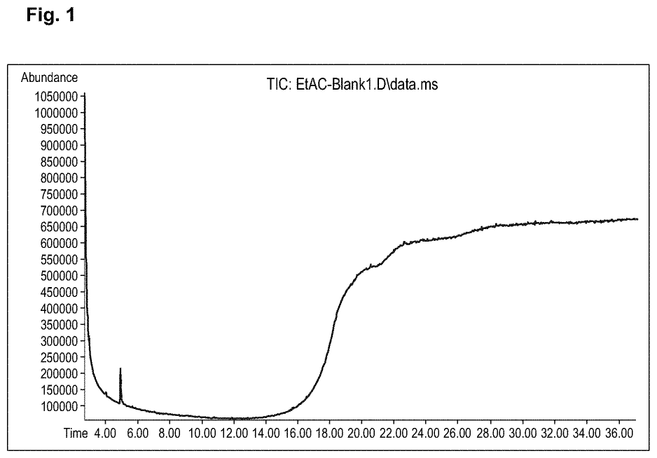

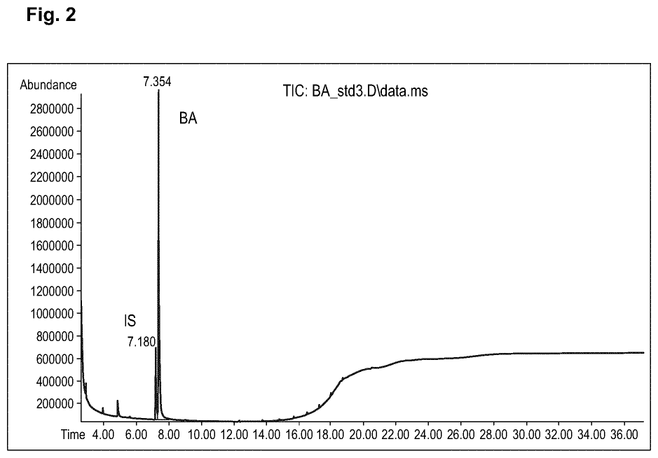

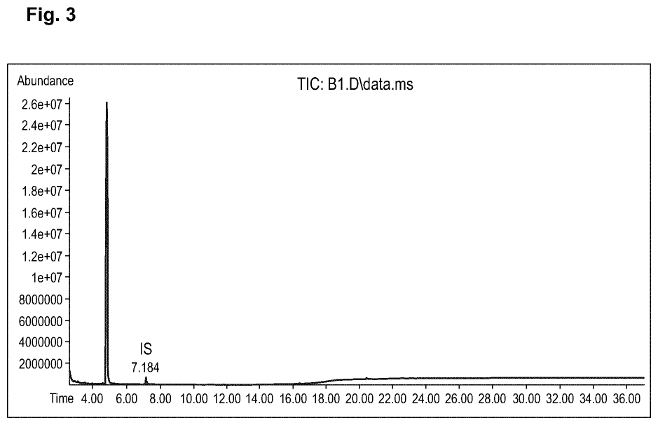

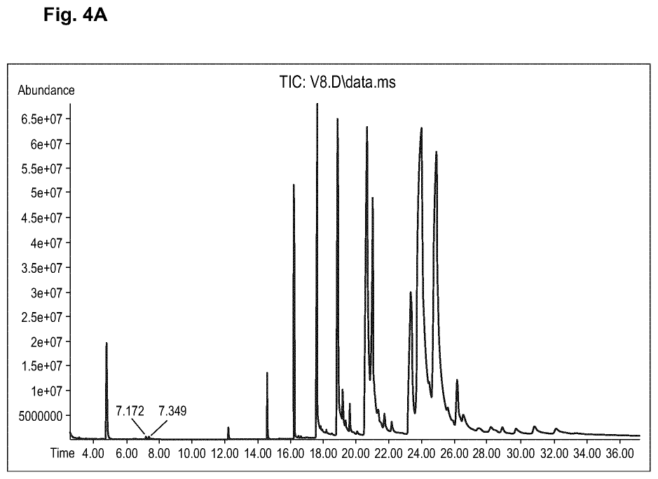

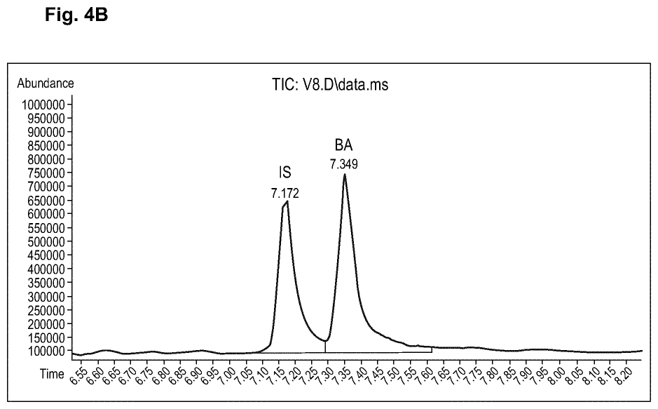

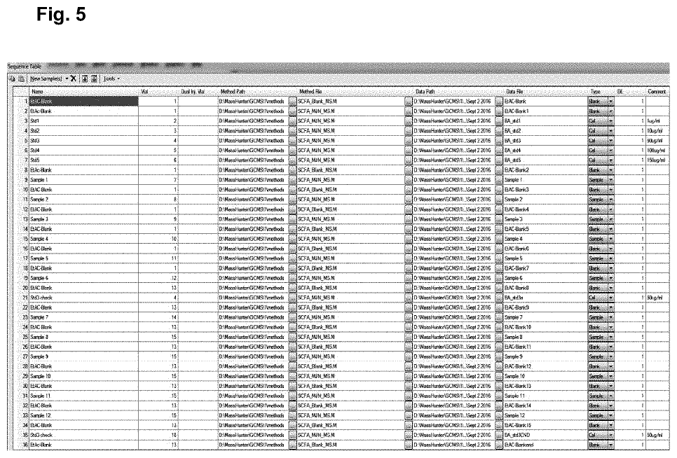

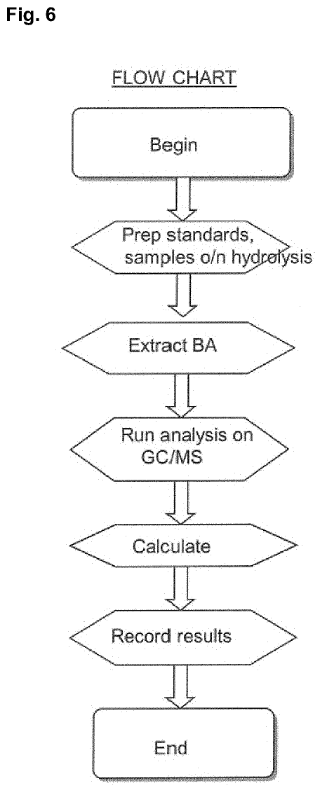

FIG. 1 illustrates an example chromatogram of ethyl acetate solvent blank. FIG. 2 illustrates an example chromatogram of an internal standard at 15 and butyric acid at 50 μg/mL. FIG. 3 illustrates an example chromatogram of NP H₂O blank through the sample preparation. FIG. 4A illustrates a chromatogram of a human milk sample and FIG. 4B illustrates the same chromatogram of a human milk sample showing the internal standard and butyric acid (BA) peaks. FIG. 5 is a sample sequence setup template. FIG. 6 illustrates a schematic process flow chart for the procedure outlined in Example 4.

### FORMULATION EXAMPLES

Formulation examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with the example, be considered to be exemplary only, with the scope and spirit of the disclosure being indicated by the claims which follow the example.

Table 15 provides an example embodiment of a peptide component including 8 peptides from Table 3.

TABLE 15
Example peptide component
Example of Selected Peptides
for Peptide Component
SEQ ID NO 5
SEQ ID NO 24
SEQ ID NO 33
SEQ ID NO 56
SEQ ID NO 64
SEQ ID NO 13
SEQ ID NO 24
SEQ ID NO 60

Table 16 provides an example embodiment of a peptide component including certain peptides from Table 3.

TABLE 16
Example peptide component
Example of Selected Peptides
for Peptide Component
SEQ ID NO 13
SEQ ID NO 24
SEQ ID NO 60
SEQ ID NO 5
SEQ ID NO 11
SEQ ID NO 22
SEQ ID NO 25
SEQ ID NO 33
SEQ ID NO 45
SEQ ID NO 46
SEQ ID NO 47
SEQ ID NO 48
SEQ ID NO 52
SEQ ID NO 34
SEQ ID NO 36
SEQ ID NO 61
SEQ ID NO 62
SEQ ID NO 64

Table 17

Table 17, illustrated below, provides an example embodiment of the nutritional profile of a nutritional composition including dietary butyrate and describes the amount of each ingredient to be included per 100 Kcal serving of nutritional composition.

TABLE 17
Nutrition profile of an example nutritional
composition including dietary butyrate

| | per 100 Kcal | |
|---|---|---|
| Nutrient | Minimum | Maximum |
| Protein Equivalent Source (g) | 1.0 | 7.0 |
| Dietary butyrate (mg) | 0.5 | 300 |
| Lactobacillus rhamnosus GG (cfu) | 1 × 10⁴ | 1.5 × 10¹² |
| Carbohydrates (g) | 6 | 22 |
| Fat (g) | 1.3 | 7.2 |
| Prebiotic (g) | 0.3 | 1.2 |
| DHA (g) | 4 | 22 |
| Beta glucan (mg) | 2.9 | 17 |
| Probiotics (cfu) | 0.5 | 5.0 |
| Vitamin A (IU) | 9.60 × 10⁵ | 3.80 × 10⁸ |
| Vitamin D (IU) | 134 | 921 |
| Vitamin E (IU) | 22 | 126 |
| Vitamin K (mcg) | 0.8 | 5.4 |
| Thiamin (mcg) | 2.9 | 18 |
| Riboflavin (mcg) | 63 | 328 |
| Vitamin B6 (mcg) | 68 | 420 |
| Vitamin B12 (mcg) | 52 | 397 |
| Niacin (mcg) | 0.2 | 0.9 |
| Folic acid (mcg) | 690 | 5881 |
| Panthothenic acid (mcg) | 8 | 66 |
| Biotin (mcg) | 232 | 1211 |
| Vitamin C (mg) | 1.4 | 5.5 |
| Choline (mg) | 4.9 | 24 |
| Calcium (mg) | 4.9 | 43 |
| Phosphorus (mg) | 68 | 297 |
| Magnesium (mg) | 54 | 210 |
| Sodium (mg) | 4.9 | 34 |
| Potassium (mg) | 24 | 88 |
| Chloride (mg) | 82 | 346 |
| Iodine (mcg) | 53 | 237 |
| Iron (mg) | 8.9 | 79 |
| Zinc (mg) | 0.7 | 2.8 |
| Manganese (mcg) | 0.7 | 2.4 |
| Copper (mcg) | 7.2 | 41 |

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. Therefore, the spirit and scope of the appended claims should not be limited to the description of the versions contained therein.

1-21. (canceled)

22. A staged feeding regimen for an infant or child, comprising the steps of:
A) providing a first nutritional composition comprising from about 4 μg/mL to about 36 μg/mL of dietary butyrate to the infant from birth to about 3 months of age;
B) providing a second nutritional composition comprising from about 6 μg/mL to about 33 μg/mL of butyrate to the infant from 3 months of age to about 6 months of age; and
C) providing a third nutritional composition comprising from about 8 μg/mL to about 40 μg/mL of butyrate to the infant from 6 months of age to about 12 months of age.

23. The staged feeding regimen according to claim 22, wherein the first nutritional composition comprises from about 7 μg/mL to about 22 μg/mL of dietary butyrate; the second nutritional composition comprises from about 17 μg/mL to about 24 μg/mL of butyrate; and the third nutritional composition comprises from about 17 μg/mL to about 22 μg/mL of butyrate.

24. The staged feeding regimen of claim 22, wherein the first nutritional composition further comprises at least one of the following selected from the group consisting of: i) up to about 0.2 mg/100 kcal of iron; ii) up to about 0.7 mg/100 kcal of zinc; iii) up to about 2 g/100 kcal of protein; iv) a protein source comprising a whey:casein ratio of at least about 72:28; v) up to 35 mg/100 kcal of inositol; and vii) from about 1.8 μg/100 kcal to about 2.0 μg/100 kcal of vitamin D.

25. The staged feeding regimen of claim 22, wherein the second nutritional composition further comprises at least one of the following selected from the group consisting of: i) from about 0.3 mg/100 kcal to about 0.6 mg/100 kcal of iron; ii) up to about 0.9 mg/100 kcal of zinc; iii) up to about 1.9 g/100 kcal of protein; iv) a protein source comprising a whey:casein ratio of about 65:44 to about 68:32; v) up to 25 mg/100 kcal of inositol; vi) up to 9.0 mg/100 kcal of butyrate; and vii) from about 1.8 μg/100 kcal to about 2.0 μg/100 kcal of vitamin D.

26. The staged feeding regimen of claim 22, wherein the third nutritional composition further comprises at least one of the following selected from the group consisting of: i) from about 0.3 mg/100 kcal to about 1.2 mg/100 kcal of iron; ii) up to about 1 mg/100 kcal of zinc; iii) up to about 1.5 g/100 kcal of protein; iv) a protein source comprising a whey:casein ratio of about 45:55 to about 56:44; v) up to 20 mg/100 kcal of inositol; vi) up to 9.0 mg/100 kcal of butyrate; and vii) from about 1.8 μg/100 kcal to about 2.0 μg/100 kcal of vitamin D.

27. The staged feeding regimen of claim 22, wherein the first nutritional composition, second nutritional composition, and third nutritional composition each further comprise docosahexaenoic acid.

28. The staged feeding regimen of claim 22, wherein the first nutritional composition, second nutritional composition, and third nutritional composition each further comprise a carbohydrate source, a fat source, and a protein source.

29. The staged feeding regimen of claim 22, wherein the first nutritional composition, second nutritional composition, and third nutritional composition each further comprise prebiotics.

30. The staged feeding regimen of claim 22, wherein the first nutritional composition, second nutritional composition, and third nutritional composition each further comprise Lactobacillus rhamnosus GG.

31. The staged feeding regimen of claim 22, wherein the first nutritional composition, second nutritional composition, and third nutritional composition each further comprise β-glucan.

32. A staged feeding regimen for an infant or child, comprising the steps of:
A) providing a first nutritional composition comprising:
up to about 0.45 mg/100 kcal of iron;
up to about 0.7 mg/100 kcal of zinc;
up to about 2 g/100 kcal of protein;
a whey:casein ratio of at least 72:28;
up to 35 mg/100 kcal of inositol;
up to 9.0 mg/100 kcal of butyrate; and/or
from about 1.8 μg/100 kcal to about 2.0 μg/100 kcal of vitamin D;
B) providing a second nutritional composition comprising:
from about 0.3 mg/100 kcal to about 0.6 mg/100 kcal of iron;
up to about 0.9 mg/100 kcal of zinc;
up to about 1.9 g/100 kcal of protein;
a whey:casein ratio of between 56:44 and 68:32;
up to 25 mg/100 kcal of inositol;
up to 9.0 mg/100 kcal of butyrate; and/or
from about 1.8 μg/100 kcal to about 2.0 μg/100 kcal of vitamin D; and
C) providing a third nutritional composition comprising:
from about 0.6 mg/100 kcal to about 1.2 mg/100 kcal of iron;
up to about 2 mg/100 kcal of zinc;
up to about 3 g/100 kcal of protein;
a whey:casein ratio of between 45:55 and 54:46;
up to 20 mg/100 kcal of inositol;
up to 9.0 mg/100 kcal of butyrate; and/or
from about 1.8 μg/100 kcal to about 2.0 μg/100 kcal of vitamin D.

33. The staged feeding regimen of claim 32, wherein the first nutritional composition comprises up to about 0.2 mg/100 kcal of iron.

34. The staged feeding regimen of claim 32, wherein the first nutritional composition comprises a whey:casein ratio of between 72:28 and 90:10.

35. A staged feeding regimen for an infant or child, comprising the steps of:
A) providing a first nutritional composition comprising no greater than 9.0 mg/100 kcal of butyrate; and
B) providing a second nutritional composition comprising at least 9.0 mg/100 kcal of butyrate.

36. The staged feeding regimen of claim 35, wherein the first nutritional composition is provided to an infant from the age of birth to three months and the second composition is provided to an infant of age three months to twelve months.

37. The staged feeding regimen of claim 35, wherein the first nutritional composition is provided to an infant from the age of birth to six months and the second composition is provided to an infant of age six months to twelve months.

38. The staged feeding regimen of claim 37 further comprising the step of:
C) providing a third nutritional composition comprising at least 9.0 mg/100 kcal of butyrate, wherein the third nutritional composition is provided to the infant after the age of twelve months.

39. The staged feeding regimen of claim 35, wherein the first nutritional composition and/or second nutritional composition further comprise sialic acid, human milk oligosaccharides, lactoferrin, or any combination thereof.

40. The staged feeding regimen of claim 39, wherein the level of sialic acid, human milk oligosaccharides, and/or lactoferrin is higher in the first nutritional composition than the second nutritional composition.
